SEQUENCE LISTING (116 sequences)

SEQ ID NO: 1 - DNA, 32 bp, Artificial Sequence
  Synthetic Construct
ttctgggatccaatggcggcaaagctgatatc

SEQ ID NO: 2 - DNA, 39 bp, Artificial Sequence
  Synthetic Construct
gatatatgaattcttagttgtttagaaaattctcgaggc

SEQ ID NO: 3 - DNA, 33 bp, Artificial Sequence
  Synthetic Construct
aaaaagcaggctgaccgtccacgtctctctgaa

SEQ ID NO: 4 - DNA, 33 bp, Artificial Sequence
  Synthetic Construct
agaaagctgggtgaaacgatgcgaccgatagtc

SEQ ID NO: 5 - DNA, 28 bp, Artificial Sequence
  Synthetic Construct
gggacaagtttgtacaaaaaagcaggct

SEQ ID NO: 6 - DNA, 29 bp, Artificial Sequence
  Synthetic Construct
ggggaccactttgtacaagaaagctgggt

SEQ ID NO: 7 - DNA, 26 bp, Artificial Sequence
  Synthetic Construct
gagaagtgggatttaaaatagaggaa

SEQ ID NO: 8 - DNA, 22 bp, Artificial Sequence
  Synthetic Construct
tgtcatcaacataggtgaccag

SEQ ID NO: 9 - DNA, 22 bp, Artificial Sequence
  Synthetic Construct
cgatagtcacggattttctgtg

SEQ ID NO: 10 - DNA, 20 bp, Artificial Sequence
  Synthetic Construct
ctcaaggagaatggtccaca

SEQ ID NO: 11 - DNA, 20 bp, Artificial Sequence
  Synthetic Construct
cgacttggccaaatgtgata

SEQ ID NO: 12 - DNA, 21 bp, Artificial Sequence
  Synthetic Construct
tggttttctgaggcatgtaaa

SEQ ID NO: 13 - DNA, 20 bp, Artificial Sequence
  Synthetic Construct
agtgcaggaacattggttgt

SEQ ID NO: 14 - DNA, 20 bp, Artificial Sequence
  Synthetic Construct
tggacagttctggagcagat

SEQ ID NO: 15 - DNA, 18 bp, Artificial Sequence
  Synthetic Construct
caactcctccgctgtgag

SEQ ID NO: 16 - DNA, 23 bp, Artificial Sequence
  Synthetic Construct
ggcaaatatctccagtattcaca

SEQ ID NO: 17 - DNA, 22 bp, Artificial Sequence
  Synthetic Construct
ggtagggcaattgttccttaga

SEQ ID NO: 18 - DNA, 20 bp, Artificial Sequence
  Synthetic Construct
aaggagcttagcctcaccac

SEQ ID NO: 19 - DNA, 20 bp, Artificial Sequence
  Synthetic Construct
gagggaagcaagaatggaac

SEQ ID NO: 20 - DNA, 20 bp, Artificial Sequence
  Synthetic Construct
gaacacgtgcaatggagttt

SEQ ID NO: 21 - DNA, 21 bp, Artificial Sequence
  Synthetic Construct
ggttccaccattgttacacct

SEQ ID NO: 22 - DNA, 19 bp, Artificial Sequence
  Synthetic Construct
aatcacagcacttgcacca

SEQ ID NO: 23 - DNA, 20 bp, Artificial Sequence
  Synthetic Construct
gagggaagcaagaatggaac

SEQ ID NO: 24 - DNA, 20 bp, Artificial Sequence
  Synthetic Construct
cgatcaaggtcaacacatgg

SEQ ID NO: 25 - DNA, 20 bp, Artificial Sequence
  Synthetic Construct
tcaaccattacccagtgtgc

SEQ ID NO: 26 - DNA, 20 bp, Artificial Sequence
  Synthetic Construct
ttccaggtdattaaycaygg

SEQ ID NO: 27 - DNA, 20 bp, Artificial Sequence
  Synthetic Construct
cataaytggagrgaytayct

SEQ ID NO: 28 - DNA, 20 bp, Artificial Sequence
  Synthetic Construct
garcaaggrcarcayatggc

SEQ ID NO: 29 - DNA, 20 bp, Artificial Sequence
  Synthetic Construct
aatcctccttchttcaagga

SEQ ID NO: 30 - DNA, 20 bp, Artificial Sequence
  Synthetic Construct
agtgcattkgggtchgtrtg

SEQ ID NO: 31 - DNA, 20 bp, Artificial Sequence
  Synthetic Construct
aatgttratgacaaargcat

SEQ ID NO: 32 - DNA, 20 bp, Artificial Sequence
  Synthetic Construct
gccatrtgytgyccttgytc

SEQ ID NO: 33 - DNA, 20 bp, Artificial Sequence
  Synthetic Construct
tccggacattgaaacttgtg

SEQ ID NO: 34 - DNA, 20 bp, Artificial Sequence
  Synthetic Construct
tcaaagaactgcttgccaac

SEQ ID NO: 35 - DNA, 20 bp, Artificial Sequence
  Synthetic Construct
cgcactcaccattctccttc

SEQ ID NO: 36 - DNA, 19 bp, Artificial Sequence
  Synthetic Construct
ggcctccaagtcctcaaag

SEQ ID NO: 37 - DNA, 25 bp, Artificial Sequence
  Synthetic Construct
tttgggaacagaaaaagttggaggt

SEQ ID NO: 38 - DNA, 25 bp, Artificial Sequence
  Synthetic Construct
catattcaaaagggaaaatcccaga

SEQ ID NO: 39 - DNA, 25 bp, Artificial Sequence
  Synthetic Construct
tggggttgtggtttattctgttgac

SEQ ID NO: 40 - DNA, 26 bp, Artificial Sequence
  Synthetic Construct
tggccaatagtagttgatacgcaaga

SEQ ID NO: 41 - DNA, 25 bp, Artificial Sequence
  Synthetic Construct
tctcgggtaagacacaagtcgagat

SEQ ID NO: 42 - DNA, 25 bp, Artificial Sequence
  Synthetic Construct
tattccaacttgcgacgtagagcat

SEQ ID NO: 43 - DNA, 24 bp, Artificial Sequence
  Synthetic Construct
ccaattgggttatttacttcgatt

SEQ ID NO: 44 - DNA, 24 bp, Artificial Sequence
  Synthetic Construct
cggcttttaacaacatattttcca

SEQ ID NO: 45 - DNA, 25 bp, Artificial Sequence
  Synthetic Construct
aacacatcaccaagatgaatccaga

SEQ ID NO: 46 - DNA, 25 bp, Artificial Sequence
  Synthetic Construct
cctctgccccaagaaatattgagat

SEQ ID NO: 47 - DNA, 24 bp, Artificial Sequence
  Synthetic Construct
agctttgtatggtagtgccaatga

SEQ ID NO: 48 - DNA, 24 bp, Artificial Sequence
  Synthetic Construct
gcggtatacgggggttaaaatcta

SEQ ID NO: 49 - DNA, 22 bp, Artificial Sequence
  Synthetic Construct
atggccaaccactctttgttac

SEQ ID NO: 50 - DNA, 22 bp, Artificial Sequence
  Synthetic Construct
acaagcaagaagaacagcgaag

SEQ ID NO: 51 - DNA, 24 bp, Artificial Sequence
  Synthetic Construct
gaaatttggttgttggcatttatc

SEQ ID NO: 52 - DNA, 25 bp, Artificial Sequence
  Synthetic Construct
tcaagatcttcatattctcattcca

SEQ ID NO: 53 - DNA, 25 bp, Artificial Sequence
  Synthetic Construct
cagctgaagtatgtttcatcccttt

SEQ ID NO: 54 - DNA, 24 bp, Artificial Sequence
  Synthetic Construct
cttgcaattgttgggactaggtaa

SEQ ID NO: 55 - DNA, 24 bp, Artificial Sequence
  Synthetic Construct
tcactaaccagtgaaaaaggttgc

SEQ ID NO: 56 - DNA, 24 bp, Artificial Sequence
  Synthetic Construct
tatacagcgaatagcaaagccaag

SEQ ID NO: 57 - DNA, 24 bp, Artificial Sequence
  Synthetic Construct
ccgcgagtgtaatatatctctcct

SEQ ID NO: 58 - DNA, 24 bp, Artificial Sequence
  Synthetic Construct
cagtttaacgcatgaagtgctagt

SEQ ID NO: 59 - DNA, 24 bp, Artificial Sequence
  Synthetic Construct
gcatcatttgtaccgtactgagtc

SEQ ID NO: 60 - DNA, 24 bp, Artificial Sequence
  Synthetic Construct
tagtggatactctgtccctgaggt

SEQ ID NO: 61 - DNA, 1026 bp, Arabidopsis thaliana
atggcggcaaagctgatatccaccggtttccgtcatactactttgccggaaaactatgtccggccaatctccgaccgtccacgtctctctgaagtctctcaactcgaagatttccctctcatcgatctctcttccactgatcgatcttttctcatccaacaaatccaccaagcttgtgcccgattcggattttttcaggtcataaatcacggagttaacaaacaaataatagatgagatggtgagtgttgcgcgtgagttctttagcatgtctatggaagaaaaaatgaagctatattcagacgatccaacgaagacaacaagattatcgacgagcttcaatgtgaagaaagaagaagtcaacaattggagagactatctaagactccattgttatcctatccacaagtatgtcaatgagtggccgtcaaaccctccttctttcaaggaaatagtaagtaaatacagtagagaagtaagagaagtgggatttaaaatagaggaattaatatcagagagcttaggtttagaaaaagattacatgaagaaagtgcttggtgaacaaggtcaacacatggcagtcaactattatcctccatgtcctgaacctgagctcacttacggtttacctgctcataccgacccaaacgccctaaccattcttcttcaagacactactgtttgcggtctccagatcttgatcgacggtcagtggttcgccgttaatccacatcctgatgcttttgtcatcaacataggtgaccagttacaggcattaagtaatggagtatacaaaagtgtttggcatcgcgctgtaacaaacacagaaaatccgagactatcggtcgcatcgtttctgtgcccagctgactgtgctgtcatgagcccggccaagcccttgtgggaagctgaggacgatgaaacgaaaccagtctacaaagatttcacttatgcagagtattacaagaagttttggagtaggaatctggaccaagaacattgcctcgagaattttctaaacaactaa

SEQ ID NO: 62 - protein, 341 aa, Arabidopsis thaliana
MAAKLISTGFRHTTLPENYVRPISDRPRLSEVSQLEDFPLIDLSSTDRSFLIQQIHQACARFGFFQVINHGVNKQIIDEMVSVAREFFSMSMEEKMKLYSDDPTKTTRLSTSFNVKKEEVNNWRDYLRLHCYPIHKYVNEWPSNPPSFKEIVSKYSREVREVGFKIEELISESLGLEKDYMKKVLGEQGQHMAVNYYPPCPEPELTYGLPAHTDPNALTILLQDTTVCGLQILIDGQWFAVNPHPDAFVINIGDQLQALSNGVYKSVWHRAVTNTENPRLSVASFLCPADCAVMSPAKPLWEAEDDETKPVYKDFTYAEYYKKFWSRNLDQEHCLENFLNN

SEQ ID NO: 63 - protein, 335 aa, Unknown
  Aquilegia species
MESSNVLLTGTRHSNLPENYVRSVSDRPRLSEVKDCENVPVIDLSVADESLLAQQIGNACKSHGFFQVINHGVNSELVEKMMEISHEFFHLPLDVKMQFYSDDPTKTMRLSTSFNLKKESVHNWRDYLRLHCHPIEKYVQEWPSVPSTFKDVVATYCKEVRKLGLRLLGSISLSLGLEEDYIEKVLGDQGQHMAVNYYPPCPEPELTYGLPRHTDPNTITILLQGQEVAGLQVLHNGKWVAVNPYPNAFVVNIGDQIQALSNGNYASVWHRATVNTDRERISVASFLCPANDAIICPAVKDGSPSMYKKFTYDEYYKKFWSGNLDQQHCLELFKE

SEQ ID NO: 64 - protein, 337 aa, Citrus sinensis
MDTKVLSSGIRYTNLPEGYVRPESERPNLSEVSECKNVPVIDLACDDRSLIVQQVADACKNYGFFQAINHEVPLETVERVLEVAKEFFNLPVEEKLKLYSDDPSKTMRLSTSFNVNKEKVHNWRDYLRLHCYPLDKYVPEWPSNPSTFKEFVSTYCSEVRGLGYRVLELISESLGLEKDYIKKVLGEQGQHMAVNFYPPCPEPELTYGLPGHTDPNALTILLQDLEVAGLQVLKDDKWVAVNPLPNAFVINIGDQLQALSNGRYKSVWHRAIVNAEKARMSVASFLCPNNDAMISPPKALTEDGSGAVYRDFTYAEYYSKFWSRNLDQEHCLELFKN

SEQ ID NO: 65 - protein, 337 aa, Coffea canephora
METKVISSGIKYTSLPESYVRPESERPRLSEVSDCQNVPVVDLGFGDRNLMVRQIGDACRDYGFFQVINHGVSKDAVDKMLETATEFFSLPVEEKLKLYSDDPSKTTRLSTSFNVKKETVHNWRDYLRLHCYPLEKYVPEWPSNPPSFKEMVSNYCVQIRELGLRLEEAIAESLGLDKECIKKVLGDQGQHMAVNYYPPCPQPDLTYGLPGHTDPNALTILLQDLNVAGLQVLRDGRWLAVKPHPDAFVVNIGDQLQALSNGIYKSVWHRAVVNADQPRLSVASFLCPCDHAVISAPKPLTADGSPVVYRDFTYAQYYKKFWSRNLDQEHCLELFKN

SEQ ID NO: 66 - DNA, 1029 bp, Cucumis sativus
atgagcagtgtgatggagatccaacttttgtgttcagggggacgtcacgagaagttgccagagaagtatgaacggcctgaatcggataggccgcggctgtcggaggtgtgttgttgggacaaggttccaataatcgacttgggatgcgaggagagagagatgattgtgaagcaagtggaggaggcctgcaagtcttacggctttttccaggttataaatcatggtgtgaggaaggaattggtggagaaagtgatagaagttggcaagcagttctttgagctgccgatggaggagaagttgaaattttattcagacgacccttccaagaccgtcagactctccacaagtttcaatgtccggaaagagcaatttcgcaactggagggattatctcagactccattgctatcctctctccaactacaccccccattggccctctaacccaccatccttcagggaaatagtgagtagttattgcaatgaagtacgaaaagttgggtacagaatagaggagctaatatcggagagcttggggctggagaaggaatacataaggaagaagttgggtgaacaaggtcagcacatggctataaattattatccgccatgtccccaaccagaactcacctacgggctccctggccatacggatcccaacgcactcaccattctccttcaggatctccatgtcgccggcctccaagtcctcaaagatggaaagtggctagcggtcaacccccaccccaatgcctttgtaatcaatataggcgaccaattgcaggcattgagcaatggggtgtacaagagcgtttggcaccgagcggtggtcaatgttgataagcccaggctgtcggtcgcttcttttctctgcccttgtgatgacgccctcattactcctgcaccgctcctctcccagccttcccccatttacagacctttcacctacgcccagtactacaatactttttggagcagaaacttggatcaacaacattgcttggaactatttaaaaaccaccctccttaa

SEQ ID NO: 67 - protein, 342 aa, Cucumis sativus
MSSVMEIQLLCSGGRHEKLPEKYERPESDRPRLSEVCCWDKVPIIDLGCEEREMIVKQVEEACKSYGFFQVINHGVRKELVEKVIEVGKQFFELPMEEKLKFYSDDPSKTVRLSTSFNVRKEQFRNWRDYLRLHCYPLSNYTPHWPSNPPSFREIVSSYCNEVRKVGYRIEELISESLGLEKEYIRKKLGEQGQHMAINYYPPCPQPELTYGLPGHTDPNALTILLQDLHVAGLQVLKDGKWLAVNPHPNAFVINIGDQLQALSNGVYKSVWHRAVVNVDKPRLSVASFLCPCDDALITPAPLLSQPSPIYRPFTYAQYYNTFWSRNLDQQHCLELFKNHPP

SEQ ID NO: 68 - protein, 337 aa, Gossypium hirsutum
MDTKVLSSGIHYSSLPESYVRPESERPRLSEVSQCDNVPVIDLGCEDRSHIVQQIALACINYGFFQVINHGVSKEAVERMLQVAHDFFGLPVEEKMKLYSDDPSKTMRLSTSFNVKKEKVHNWRDYLRLHCYPLHKYVPEWPSNPPSFKQIVSDYCVQVRELGYRLQELISESLGLEKDYIKKVLGEQGQHMAVNYYPPCPEPELTYGLPGHTDPNALTILLQDLQVAGLQVLKDGKWLAVNPQTNAFVINIGDQLQALSNGTYKSVWHRAIVNTDKPRMSVASFLCPYDHALISPAKPLTQHGCGAVYRDFTYAEYYSKFWGRNLDQEHCLELFKN

SEQ ID NO: 69 - DNA, 1014 bp, Lactuca sativa
atggccgcaaaagtcatctccagtggattccggtatactactctaccggagagctacgtccgtccggttaacgacagacctaacctatctcaagtttccgattgcaacgacgttcctgttattgacatcggttgtggtgatagacaactcataagccaacaaattggcgatgcttgtagaagatacggttttttccaggtgattaatcatggtgtgcctgatgaaatagtggagaaaatgcaacaagtaggtagggagtttttcctgttgcctgtggaagagaagatgaagctttactcagaggatccatcgaagacgatgaggctatccaccagctttaacgtccaaaaagaacaaattcataactggcgagattatctccgccttcactgttatcctctggatcaatacagtcctgaatggccttcaaatccttcttatttcaaggaatatgttggtaattattgtacagcagtgcgaaatttaggaatgagaatattagaatcaatatcagaaagtttagggttacaaaaagaagaaataaaaactatattaggcgatcaaggtcaacacatggccatcaaccattacccagtgtgccctgagcccgagctaacctacgggctacccgggcacacagaccccaatgctctcaccatccttctacaggacacactggtctctggtcttcaggttctcaaagatggcaaatggttagccgttaaaccacaccctaatgcgtttgtaattaacattggtgatcagttagaggcggtgagtaatggtgaatataaaagtgtatggcatcgagctgtggttaactcagacaacccgcgaatgtctatagcttcgtttttgtgtccttgtaatgacaccgttattagggctcctaaagaaataataaaggaaggatcgaaacctgttttcaaagaatttacttatgcagaatactacgcgaagttttggacaagaaaccttgatcaagaacattgcttagaattcttcaagaactag

SEQ ID NO: 70 - protein, 337 aa, Lactuca sativa
MAAKVISSGFRYTTLPESYVRPVNDRPNLSQVSDCNDVPVIDIGCGDRQLISQQIGDACRRYGFFQVINHGVPDEIVEKMQQVGREFFLLPVEEKMKLYSEDPSKTMRLSTSFNVQKEQIHNWRDYLRLHCYPLDQYSPEWPSNPSYFKEYVGNYCTAVRNLGMRILESISESLGLQKEEIKTILGDQGQHMAINHYPVCPEPELTYGLPGHTDPNALTILLQDTLVSGLQVLKDGKWLAVKPHPNAFVINIGDQLEAVSNGEYKSVWHRAVVNSDNPRMSIASFLCPCNDTVIRAPKEIIKEGSKPVFKEFTYAEYYAKFWTRNLDQEHCLEFFKN

SEQ ID NO: 71 - protein, 338 aa, Medicago truncatula
MDTKVLSSGIHYSKLPESYIRPESDRPCLSQVSEFENVPIIDLGSHNRTQIVQQIGEACSSYGFFQVVNHGVPLEELKKTAEVAYDFFKLPVEEKMKLYSDDPTKTMRLSTSFNVNKEEVHNWRDYLRLHCYPLDNYVPEWPSNPPSFKETVANYCKEVRELGLRIEEYISESLGLEKDYLRNALGEQGQHMAVNYYPPCPQPELTYGLPGHTDPNALTILLQDLHVAGLQVLKDGKWLAINPIPDAFVINIGDQLQALSNGLYKSVWHRAIVNAEKPRLSVASFLCPDNEALICPAKPLTEDGSGAVYRGFTYPEYYSKFWSRDLEKEHCLEFFKNN

SEQ ID NO: 72 - protein, 342 aa, Oryza sativa
MAAEAEQQHQLLSTAVHDTMPGKYVRPESQRPRLDLVVSDARIPVVDLASPDRAAVVSAVGDACRTHGFFQVVNHGIDAALIASVMEVGREFFRLPAEEKAKLYSDDPAKKIRLSTSFNVRKETVHNWRDYLRLHCYPLHQFVPDWPSNPPSFKEIIGTYCTEVRELGFRLYEAISESLGLEGGYMRETLGEQEQHMAVNYYPQCPEPELTYGLPAHTDPNALTILLMDDQVAGLQVLNDGKWIAVNPQPGALVINIGDQLQALSNGKYRSVWHRAVVNSDRERMSVASFLCPCNSVELGPAKKLITDDSPAVYRNYTYDEYYKKFWSRNLDQEHCLELFRT

SEQ ID NO: 73 - protein, 342 aa, Oryza sativa
MADQLISTADHDTLPGNYVRPEAQRPRLADVLSDASIPVVDLANPDRAKLVSQVGAACRSHGFFQVLNHGVPVELTLSVLAVAHDFFRLPAEEKAKLYSDDPAKKIRLSTSFNVRKETVHNWRDYLRLHCYPLHRYLPDWPSNPPSFREIISTYCKEVRELGFRLYGAISESLGLEQDYIKKVLGEQEQHMAVNFYPKCPEPELTFGLPAHTDPNALTILLMDQQVAGLQVLKEGRWIAVNPQPNALVINIGDQLQALSNGRYKSVWHRAVVNSDKARMSVASFLCPCNDVLIGPAQKLITDGSPAVYRNYTYDEYYKKFWSRNLDQEHCLELFRTTPTDTS

SEQ ID NO: 74 - protein, 340 aa, Oryza sativa
MATTQLLSTVEHRETLPEGYARPESDRPRLAEVATDSNIPLIDLASPDKPRVIAEIAQACRTYGFFQVTNHGIAEELLEKVMAVALEFFRLPPEEKEKLYSDEPSKKIRLSTSFNVRKETVHNWRDYLRLHCHPLEEFVPEWPSNPAQFKEIMSTYCREVRQLGLRLLGAISVSLGLEEDYIEKVLGEQEQHMAVNYYPRCPEPDLTYGLPKHTDPNALTILLPDPHVAGLQVLRDGDQWIVVNPRPNALVVNLGDQIQALSNDAYKSVWHRAVVNPVQERMSVASFMCPCNSAVISPARKLVADGDAPVYRSFTYDEYYKKFWSRNLDQEHCLELFKGQ

SEQ ID NO: 75 - protein, 338 aa, Populus trichocarpa
MDTKVLSSGIQYTNLPASYVRPESERPRLWEVSTCENVPVIDLGCQERDQIVQQVGDACKNYGFFQVINHGVSLEAVEKMLGVAHDFFSLPVEEKLKLYSDDPSKTMRLSTSFNVNKEKVHNWRDYLRLHCYPLDKYAPEWPSKPPPFKDIVSSYCIQVRELGFRIQELISESLGLEKDHVKNVLGEQGQHMAVNFYPPCPEPELTFGLPGHTDPNALTILLQDQSVAGLQVLKDGKWVAVDPHPDAFVINIGDQLQALSNGRYKSVWHRAITNTDKARMSVASFLCPYDNALITPPKALTDDGTGAVYRDFTYAEYYKKFWSRDLDQEHCLELFKNK

SEQ ID NO: 76 - protein, 338 aa, Populus trichocarpa
MDTKVISSGVHYTNLPASYVRPESERPRLSEVSTCEDVPVIDLGCQDRNQIVQQVGDACEHYGFFQVINHGVSLEAVEKMLGVAHDFFSLPVEEKLKLYSDDPSKTMRLSTSFNVNKEKVHNWRDYLRLHCYPLDKYVPEWPSNPPPFKEIVRSYSIQVRELGFRIQELISESLGLEKDHIKNVLGEQGQHMAVNFYPPCPEPELTYGLPAHTDPNALTILLQDLSVAGLQVLLKDGKWVAVNPHPDAFVINIGDQLQALSNGRYKSVWHRAITNTDKARMSVASFLCPFDNALITPPKALTDDGTGAIYRDFTYAEYYKKFWSRNLDQEHCLELFKN

SEQ ID NO: 77 - protein, 337 aa, Solanum lycopersicum
METKVISSGINHSTLPQSYIRPESDRPRLSEVVDCENVPIIDLSCGDQAQIIRQIGEACQTYGFFQVINHGVPKEVVEKMLGVAGEFFNLPVEEKLKLYSDDPSKTMRLSTSFNVKKETVHNWRDYLRLHCYPLEKYAPEWPSNPSSFREIVSRYCREIRQLGFRLEEAIAESLGLDKECIKDVLGEQGQHMAINYYPPCPQPELTYGLPAHTDPNSLTILLQDLQVAGLQVLKDGKWLAVKPQPDAFVINLGDQLQAVSNGKYRSVWHRAIVNSDQARMSVASFLCPCDSAKISAPKLLTEDGSPVIYQDFTYAEYYNKFWSRNLDQQHCLELFKN

SEQ ID NO: 78 - protein, 342 aa, Solanum lycopersicum
MTTTSVLSSGFNHSTLPQSYVRPESQRPCMSEVVDSDDLVPVIDMSCTNRNVIVHQIGEACRLYGFFQVINHGVSKKVIDEMLGVSHEFFKLPVEEKMKLYSDDPSKTMRLSTSFNVKKETVHNWRDYLRLHCYPLDKYAPEWPSNPPSFREIVSKYCMEVRELGYRLEEAISESLGLEKDCIKNVLGEQGQHMAINFYPQCPQPELTYGLPAHTDPNAITILLQDLQVAGLQVLKDGKWLSIKPQPNAFVINLGDQLEALSNGKYKSIWHRAIVNSDKARMSVASFLCPNDCSIISAPKTLTEDGSSAIYRHFTYAEYYEKFWSRNLDQEYCLELFKNDGT

SEQ ID NO: 79 - protein, 336 aa, Sorghum bicolor
MAEQLLSTAVHDTLPGSYVRPESQRPRLAEVVTGARIPVVDLGSPDRAAVVAAIGDACRSHGFFQVLNHGVHADLVAAVMAVGRAFFRLSPEEKAKLYSDDPARKIRLSTSFNVRKETVHNWRDYLRLHCHPLDEFVPDWPSNPPDFKDTMSTYCKEVRELGFRLYAAISESLGLEASYMKETLGEQEQHMAVNFYPPCPEPELTYGLPAHTDPNALTILLMDQDVAGLQVLHGGKWVAVNPQPGALIINIGDQLQALSNGQYRSVWHRAVVNSDRERMSVASFLCPCNHVVLGPAKKLVTEDTPAVYRSYTYDEYYKKFWSRNLDQEHCLELFRT

SEQ ID NO: 80 - DNA, 1020 bp, Spinacia oleracea
atggcaaacaagatattatccaccggaattccttacaaaaccctccccgaaagctacatccgacccgaaaatgagaggcccaacttatctcaagtctccgattgcgagaatgtccctgttattgacttgggtgccaaagaccgtactcaaacaatccaccaagtcttcaatgcttgtaaaaattacgggtttttccaggtgattaatcatggggtgtcaaaggaattagcggagaagatgcaaaaggtagctcgagagttcttcgatatgtcggttgaggaaaaaatgaaattatatagtgacgatccaactaaaacactaagattgtctacaagttttaacgttaacaaagaggaagttcataattggagagattatcttaggctccattgttggcctcttgagcaatatgtccccgaatggccttctaaccccccttccttcaaggaaatagtgagcaagtacataaaagaagttagggaacttggtttcagagtccaagaactaatatcagagagtttagggttggagaaagattacataaagaatgtcctaggagatcaaggacaacacatggctcttaattattaccctgagtgcccggagccagagatgacatacgggttgccgggtcatactgaccctaatgcccttaccatccttctccaagacttgcaagtatctggccttcaaatttttaaggatggtaaatggcttgctgtcaaacctcaacctgatgcttttgtcattaacattggtgatcaattgcaggcattaagtaacggtatatacaagagtgtatggcacagagcagttgtgaacacagataagccaagattatcagtagcttcattcctctgccccgccaatgatgcgttgataagcgcgccaacacctctgaccgccaacggatcaccggctgtatatagagactatacgtatcctgagtactacaagactttctggagtaggaacttggaccaagagcactgcttggagctttttaaaaaccaaacctag

SEQ ID NO: 81 - protein, 339 aa, Spinacia oleracea
MANKILSTGIPYKTLPESYIRPENERPNLSQVSDCENVPVIDLGAKDRTQTIHQVFNACKNYGFFQVINHGVSKELAEKMQKVAREFFDMSVEEKMKLYSDDPTKTLRLSTSFNVNKEEVHNWRDYLRLHCWPLEQYVPEWPSNPPSFKEIVSKYIKEVRELGFRVQELISESLGLEKDYIKNVLGDQGQHMALNYYPECPEPEMTYGLPGHTDPNALTILLQDLQVSGLQIFKDGKWLAVKPQPDAFVINIGDQLQALSNGIYKSVWHRAVVNTDKPRLSVASFLCPANDALISAPTPLTANGSPAVYRDYTYPEYYKTFWSRNLDQEHCLELFKNQT

SEQ ID NO: 82 - protein, 338 aa, Vitis vinifera
MESKVLSTGIRYLTLPQSYIRPEPERPRLSQVSECKHVPIIDLGKDVNRAQLIQHIADACRLYGFFQVINHGVAAEMMEKMLEVADEFYRLPVEEKMKLYSDDPTKTMRLSTSFNVNKEKVHNWRDYLRLHCYPLDQYTPEWPSNPPSFKEIVSSYCKEVRELGFRLQEMISESLGLEKDHIKNVFGEQGQHMAVNYYPPCPQPELTYGLPGHTDPNALTILLQDLRVAGLQVLKDGTWLAIKPHPGAFVVNIGDQLQAVSNGKYKSVWHRAVVNAESERLSVASFLCPCNDAVIGPAKPLTEDGSAPIYKNFTYAEYYKKFWGRDLDQEHCLELFKN

SEQ ID NO: 83 - protein, 336 aa, Zea mays
MAEHLLSTAVHDTLPGSYVRPEPERPRLAEVVTGARIPVVDLGSPDRGAVVAAVGDACRSHGFFQVVNHGIHAALVAAVMAAGRGFFRLPPEEKAKLYSDDPARKIRLSTSFNVRKETVHNWRDYLRLHCHPLDEFLPDWPSNPPDFKETMGTYCKEVRELGFRLYAAISESLGLEASYMKEALGEQEQHMAVNFYPPCPEPELTYGLPAHTDPNALTILLMDPDVAGLQVLHAGQWVAVNPQPGALIINIGDQLQALSNGQYRSVWHRAVVNSDRERMSVASFLCPCNHVVLGPARKLVTEDTPAVYRNYTYDKYYAKFWSRNLDQEHCLELFRT

SEQ ID NO: 84 - protein, 346 aa, Zingiber officinale
MADMLLSIGEHDTMPRNYVRPENERPHLDNVIADANIPVVDFGAPDKSQIISQIEKACRLYGFFQVVNHGIAAELIKKVLAIALEFFRLPQEEKAKLYSDDPAKKIRLSTSFNVRKETVHNWRDYLRLHCYPLEEFIPDWPSNPSSFKDVFGSYCQQVRKLGFRILGIISLSLGLEEEYLVRVLGEQEQHMAVNYYPKCPEPELTYGLPAHTDPNALTILLQDPHVSGLQVHKDGKWIAVDPKPNAFVINIGDQLQALSNGRYKSVWHRAVVNSNKERMSVASFLCPCNSVLISPPEKLIADGCPAVYRSYTYDEYYKKFWSRNLDQEHCLELFKKERETCPDAPT

SEQ ID NO: 85 - DNA, 22 bp, Artificial Sequence
  Synthetic Construct
caccatggcggcaaagctgata

SEQ ID NO: 86 - DNA, 21 bp, Artificial Sequence
  Synthetic Construct
gacaaacacaaaggccaaaga

SEQ ID NO: 87 - DNA, 24 bp, Artificial Sequence
  Synthetic Construct
caccatgagcagtgtgatggagat

SEQ ID NO: 88 - DNA, 20 bp, Artificial Sequence
  Synthetic Construct
tgggccaaaaagtttatcca

SEQ ID NO: 89 - DNA, 27 bp, Artificial Sequence
  Synthetic Construct
caccatggcaaacaagatattatccac

SEQ ID NO: 90 - DNA, 22 bp, Artificial Sequence
  Synthetic Construct
ttgctgcctacaaaagtacaaa

SEQ ID NO: 91 - DNA, 24 bp, Artificial Sequence
  Synthetic Construct
caccatggccgcaaaagtcatctc

SEQ ID NO: 92 - DNA, 22 bp, Artificial Sequence
  Synthetic Construct
catggaaacacatattccttca

SEQ ID NO: 93 - DNA, 28 bp, Artificial Sequence
  Synthetic Construct
caccatggaaaccaaagttatttctagc

SEQ ID NO: 94 - DNA, 20 bp, Artificial Sequence
  Synthetic Construct
gggacatccctatgaaccaa

SEQ ID NO: 95 - DNA, 1013 bp, Solanum lycopersicum
atggaaaccaaagttatttctagcggaatcaaccactctactcttcctcaaagttacatccgacccgaatccgatagaccacgtctatcggaagtggtcgattgtgaaaatgttccaataattgacttaagttgcggagatcaagctcaaataattcgtcaaattggagaagcttgtcaaacttatggtttctttcaggtaattaatcatggtgtaccaaaggaagttgtagagaaaatgctaggggtagctggggaatttttcaatttaccagtagaagagaaactaaaattatattcagatgatccttcaaagaccatgagattatcaacaagttttaatgttaaaaaggagacagttcataattggagagattatctcagacttcattgttatcctctagagaagtatgctcctgaatggccttctaatccatcatctttcagggaaatcgtgagcagatattgcagggaaattcgtcaactcggatttagattagaagaagccatagcagaaagcctggggttagataaagagtgtataaaagatgtattgggtgaacaaggacaacatatggctatcaattattatcctccttgtccacaaccagaacttacttatgggcttccggcccatactgatccaaattcacttacaattcttcttcaagacttgcaagttgcgggtcttcaagttcttaaagatggcaaatggttagctgtaaaacctcaacctgacgcctttgtcattaatcttggggatcaattgcaggcagtaagtaacggtaagtacagaagtgtatggcatcgagctattgtgaattcagatcaagctaggatgtcagtggcttcgtttctatgtccgtgtgatagcgcgaaaatcagtgcaccaaagctgctgacagaagatggatctccagtgatttatcaagactttacgtatgctgagtattacaacaagttctggagcaggaatttggaccagcaacattgtttggaacttttcaagaataa

SEQ ID NO: 96 - protein, 337 aa, Solanum lycopersicum; identical to SEQ ID NO: 77

SEQ ID NO: 97 - DNA, 1014 bp, Nicotiana benthamiana
atggaagcaaaagttctttccagcggaatccgccactctactatccctcaaagttacatccgccctcaatccgataggccgcgcctttctgaagttgctgattgtgaaaacgttccagtagttgatataggttgcggtgatagaaaccttattgttcatcaaattggtgaagcctgtcgtctttatggttttttccaggtaattaatcatggtgtaccaaagaatttaatagacgaaatgctagagatagctggggaattttttaggcttccagttgaagagaagttgaaattgtactcagatgacccatcgaagacgatgagattgtcgactagttttaatgtgaaaaaggagaaggttcacaattggagagattatctcagacttcattgttatcctcttgaaaattacgctcctgaatggccttccaatccttcctctttcagggaaatcgtgagcagatattgcatggaagttcgacaactcgggttcagattgcaggaagccatagcagagagcctaggcttagagaaagagtgtataaaggatgtattgggcgaacaaggtcaacacatggctatcaatttctatcctccttgtccacaaccagaactcacttatgggctgccagcacatactgatccaaatgcccttacaattcttcttcaagacttagaagtagctggtcttcaagttcttaaagatggcgaatggttggccgtcaagcctcaaccagatgcctttgtcattaatcttggtgatcaactgcaggcagtgagtaatgggagatacaaaagcgtatggcatcgagctattgtaaattcagacaaagccaggttgtcagtggcttcgttcctttgtccgtgcgatagcgcgaaaatcagtgctccaaagctcctcactgaagatggatctcctgtcatttatcaggactttacctatgctgagtattacaaaaagttctggagcaggaatttggaccaggaacattgtttggaacttttcaagaactaa

SEQ ID NO: 98 - protein, 337 aa, Nicotiana benthamiana
MEAKVLSSGIRHSTIPQSYIRPQSDRPRLSEVADCENVPVVDIGCGDRNLIVHQIGEACRLYGFFQVINHGVPKNLIDEMLEIAGEFFRLPVEEKLKLYSDDPSKTMRLSTSFNVKKEKVHNWRDYLRLHCYPLENYAPEWPSNPSSFREIVSRYCMEVRQLGFRLQEAIAESLGLEKECIKDVLGEQGQHMAINFYPPCPQPELTYGLPAHTDPNALTILLQDLEVAGLQVLKDGEWLAVKPQPDAFVINLGDQLQAVSNGRYKSVWHRAIVNSDKARLSVASFLCPCDSAKISAPKLLTEDGSPVIYQDFTYAEYYKKFWSRNLDQEHCLELFKN

SEQ ID NO: 99-106 - skipped (8 entries)

SEQ ID NO: 107 - DNA, 3003 bp, Arabidopsis thaliana
catttttctataaatccaaactaacatctactttctttaaatctataaccctaaacacttttttaaactcaaaccgatatataattttgtttaattttaaatctaaactctagtgacttatttataaacccaaacctaaaaataatttcgttttattgtaaatttaaactctaatttatatttataaatctaaactgacttataattttgtttaattgtaaaatctaaattttaaatataattaatcttgtttaattaaaagtatacagatttgttattttagtttattatataatatgatataataactagtttaaattaaaagtaagagtttattcttagaggtaaatgcaagtattgtccgaaaaaacaaatctaattcaagtagtgtccgaaaaaaaattctaactagtttgatagttaaaattttgatttaaaaaaggaaaaaaatcaaacaagatattaattagaagtgtgagacacggcacaagagtcacatgagtgtacgtacttatcaagattgactctgtctgagtctgaagtcccaaaccatgatggcaccacttccacatacgatcgtgccccgtattttggatagaatacggacagtggttttcgtttggacacgtgtcctgctttatctcttcgtcgccccaaaaaataccacaatgtcttatctcaaccacacgtgttctgcttatcccaacctcacaatttgtaccaaaatacacactttgcatggaagattttctaattatacaactcacattattcgaatttaaatttcgattttttagtttcaagaaaatcattctttgatgggtacttgtcttatttaacaggttgtatacttgtattcattgttctgccaaatgaaaataaaaatgaaaatgatgttcattgtttaataaaagtactaagataacaatcacgacaaatttctgtctagttcattaaatatttaatcaaactctaaacgattttcaaacaatttttataattcaaaaaataagttacatatctttgtttaacataatataataaaaataacatgaataaattattttaacataaaaaattcagtttttcaaaaataagtttagaagtttacgttctaaaataaggtaaaatatgaatgctgttttaagacgcaatctagataattttttttaataaaaaccgagatacatttaaatctatctaaataacttataactacctaattgttacataatctaccaatttaactctatgtaaaataaaactgattttagtaacatttaagcagtacgagaatgctagcgcctaattaaacgatcttctaatccactttcttgaatatttgttttaactaaatctaaacaaaaatatagttatataaccacaaatattaatgaaatttaaacttatagtaactgaaatacccaaaactaaaaaaaaaaaccaaaattataataattataaataagaagatattagtttatgtttacaatcgaaataatcaaataaatgattgtctttatttaggactacgatcaagaaccgaatgggcttttccaaaccaaaccgagatttgaattttatggtgcggattcggttaactggagaatagctatcaacaacaatttaaaatagatttagctagatcggtttggttcggttcgttttgtattctctgtcactcctcacaatcgcttatattttatattgtatgtttaaaagtcaacatcgaaatattgtacgttagtatgtcacttatgataatgtttattcgtaaacacaatttgaaaaggtcaaagaaagaggaaagatagttaatcaagcccttgttgtcaaaaataattattttatttactgtcatcgtaatgtttatcaatgcagttattaatctcatttttttctcttccgaagtcgacgaacaataaaaaaaaccaatctcattcgaagtacttattactgatatgatgctgagctgacacagtcgtaagccttggacaacaatcattcatgacgtcactgctgtgacgctagaatgatgacattatatcaatgtttttttgtctgaattttgttatggtaaaaataatgaaaatgtagagcttgagtattttgattttcgttttattgtaaactagctgaatctgaatcttgagcagttaattaatttcgtaatttattaattctattctgactttttaaaatataatatatattaactttggtagatgcttaaggtaattcttttttaataaataagatggttagagtatcttaaagttagcttataagaaaatcggaaaaattacttttggtgggttaattgtttctgtttgaagtaatgtgtgtagatttttcttatgaatttagattaaaaactatttgtttttcagatgttttaagaaaaaaattgtcattcatagcttgtccattcttacataccttaataagaaaaattataaagttttgtggattcacggaagctaatctaggttatgtatttgcccaaaaaataatctaggttttgttatggaattaagaaggaaaaaaaaattgagataaatagtatataaaaacaatttaaactaagtattattagcttaattgataaagattttaggtgaaacttaaaaatagttggttaaagagattacaaacattaaccaaattaaccaagaacctcctagtatttaaaaaaaacacttaaaaatatccaaacatttaattttttaatcataaatcttataaaacccacagctgtcctttcgaaaatccactatattcggtggattaagaattaaaaatcattcgaataatatgcatacttatataacaaaaacaattcacttgaaaacataatcaattgagagtaggaccgagtaacactgcattgttttatatatatcatcgatgcacatcgcatacataatatactcaaagtcgagccttccttcctttatctcttataccctttttgattcttcttcaattttctgacatcaaatg

SEQ ID NO: 108 - DNA, 24 bp, Artificial Sequence
  Synthetic Construct
caggtttatggcatatctcacgtc

SEQ ID NO: 109 - DNA, 24 bp, Artificial Sequence
  Synthetic Construct
tgataccagacgttgcccgcataa

SEQ ID NO: 110 - DNA, 25 bp, Artificial Sequence
  Synthetic Construct
tcacgggttggggtttctacaggac

SEQ ID NO: 111 - DNA, 24 bp, Artificial Sequence
  Synthetic Construct
atgtccaagtccaatagccacaag

SEQ ID NO: 112-114 - skipped (3 entries)

SEQ ID NO: 115 - protein, 338 aa, Glycine max
MDIKVLSSGVQYSNLPESYIRPESERPRLSEVSECEDVPIIDLGSQNRAQIVHQIGEACRNYGFFQVINHGVALEAAKEMEEVAHGFFKLPVEEKLKLYSEDTSKTMRLSTSFNVKKETVRNWRDYLRLHCYPLEKYAPEWPSNPPSFKETVTEYCTIIRELGLRIQEYISESLGLEKDYIKNVLGEQGQHMAVNYYPPCPEPELTYGLPGHTDPNALTILLQDLQVAGLQVLKDGKWLAVSPQPNAFVINIGDQLQALSNGLYKSVWHRAVVNVEKPRLSVASFLCPNDEALISPAKPLTEHGSEAVYRGFTYAEYYKKFWSRNLDQEHCLELFKNK

SEQ ID NO: 116 - protein, 338 aa, Glycine max
MDTKVLSSGVQYSNLPESYIRPESERPRLSEVSECEDVPIIDLGCQNRAQIVHQIGEACRNYGFFQVINHGVALEAAKEMAEVAHGFFKLPVEEKLKLYSEDPSKTMRLSTSFNVKKETVHNWRDYLRLHCYPLDKYAPEWPSNPPSFKETVTEYCTLVRELGLRIQEYISESLGLEKDYIKNVLGEQGQHMAVNYYPPCPEPELTYGLPGHTDPNALTILLQDLQVCGLQVLKNGKWLAVNPQPNAFVINIGDQLQALSNGLYKSVWHRAVVNVEKPRLSVASFLCPNDEALISPAKPLTEGGSEAIYRGFTYAEYYKKFWSRNLDQEHCLEFFKNK

(19) United States
(12) Patent Application Publication
(10) Pub. No.: US 2021/0071195 A1
(43) Pub. Date: Mar. 11, 2021

(54) SOYBEAN PLANTS RESISTANT TO PHYTOPHTHORA SOJAE

(71) Applicant: SciENZA Biotechnologies 3 B.V., Enkhuizen (NL)

(72) Inventor: Tieme ZEILMAKER, Amersfoort (NL)

(73) Assignee: SciENZA Biotechnologies 3 B.V., Enkhuizen (NL)

(21) Appl. No.: 16/642,257

(22) PCT Filed: Aug. 27, 2018
(86) PCT No.: PCT/EP2018/073019

§ 371 Date: Feb. 26, 2020

Related U.S. Application Data
(60) Provisional application No. 62/551,716, filed on Aug. 29, 2017.

(51) Int. Cl. C12N 15/82; C12N 9/02

(52) U.S. Cl. CPC C12N 15/8282; C12N 9/0073

(57) ABSTRACT

The present disclosure relates to a soybean plant, which is resistant to a pathogen of viral, bacterial, fungal or oomycete origin, wherein the soybean plant has a reduced level, reduced activity or complete absence of DMR6 protein as compared to a soybean plant that is not resistant to the said pathogen, in particular organisms of the kingdom Fungi or the phylum Oomycota. The present disclosure further relates to a method for obtaining a soybean plant, which is resistant to a pathogen of viral, bacterial, fungal or oomycete origin, comprising reducing the endogenous level or activity of DMR6 protein in the soybean plant. In addition, the present disclosure relates to the use of a DMR6 promoter for providing disease resistant soybean plants.

### FIELD OF THE INVENTION

The present disclosure relates to disease resistant plants, in particular plants resistant to organisms of the kingdom Fungi and the phylum Oomycota, the oomycetes. The present disclosure further relates to plant genes conferring disease resistance and methods of obtaining such disease resistant plants for providing protection to Oomycota pathogens.

### BACKGROUND

Resistance of plants to fungal and oomycete pathogens has been extensively studied, for both pathogen specific and broad resistance. In many cases resistance is specified by dominant genes for resistance. Many of these race-specific or gene-for-gene resistance genes have been identified that mediate pathogen recognition by directly or indirectly interacting with avirulence gene products or other molecules from the pathogen. This recognition leads to the activation of a wide range of plant defense responses that arrest pathogen growth.

In plant breeding there is a constant struggle to identify new sources of mostly monogenic dominant resistance genes. In cultivars with newly introduced single resistance genes, protection from disease is often rapidly broken, because pathogens evolve and adapt at a high frequency and regain the ability to successfully infect the host plant.

Therefore, the availability of new sources of disease resistance is highly needed.

Alternative resistance mechanisms act for example through the modulation of the defense response in plants, such as the resistance mediated by the recessive mlo gene in barley to the powdery mildew pathogen Blumeria graminis fsp. hordei. Plants carrying mutated alleles of the wildtype MLO gene exhibit almost complete resistance coinciding with the abortion of attempted fungal penetration of the cell wall of single attacked epidermal cells. The wild type MLO gene thus acts as a negative regulator of the pathogen response. This is described in WO9804586.

Other examples are the recessive powdery mildew resistance genes, found in a screen for loss of susceptibility to Erysiphe cichoracearum. Three genes have been cloned so far, named PMR6, which encodes a pectate lyase-like protein, PMR4 which encodes a callose synthase, and PMR5 which encodes a protein of unknown function. Both mlo and pmr genes appear to specifically confer resistance to powdery mildew and not to oomycetes such as downy mildews.

Broad pathogen resistance, or systemic forms of resistance such as SAR, has been obtained by two main ways. The first is by mutation of negative regulators of plant defense and cell death, such as in the cpr, lsd and acd mutants of Arabidopsis. The second is by transgenic overexpression of inducers or regulators of plant defense, such as in NPR1 overexpressing plants.

The disadvantage of these known resistance mechanisms is that, besides pathogen resistance, these plants often show detectable additional and undesirable phenotypes, such as stunted growth or the spontaneous formation of cell death.

### BRIEF SUMMARY

It is an object of the present disclosure to provide a form of resistance that is broad, durable and not associated with undesirable phenotypes.

In the research that led to the present disclosure, an Arabidopsis thaliana mutant screen was performed for reduced susceptibility to the downy mildew pathogen Hyaloperonospora parasitica. EMS-mutants were generated in the highly susceptible Arabidopsis line Ler eds1-2.

Eight downy mildew resistant (dmr) mutants were analyzed in detail, corresponding to 6 different loci. Microscopic analysis showed that in all mutants H. parasitica growth was severely reduced. Resistance of dmr3, dmr4 and dmr5 was associated with constitutive activation of plant defense. Furthermore, the dmr3 and dmr4, but not dmr5 mutants, were also resistant to Pseudomonas syringae and Golovinomyces orontii.

In contrast, enhanced activation of plant defense was not observed in the dmrl , dmr2, and dmr6 mutants. The results of this research have been described in Van Damme et al. (2005) Molecular Plant-Microbe Interactions 18(6) 583-592. This article does not disclose the identification and characterization of the DMR genes.

The dmr6 mutant was identified in a loss-of-susceptibility screen in the Arabidopsis

Ler eds1-2 background. The DMR6 gene now has been cloned and characterized. Thus, it was found that DMR6 is the gene At5g24530, encoding for an oxidoreductase (DNA and amino acid sequence are depicted in FIG. 2). Oxidoreductases are enzymes that catalyze the transfer of electrons from one molecule, the oxidant, to another, the reductant. According to the present disclosure, it has been found that lack of a functional DMR6 protein results in downy mildew resistance.

The present disclosure thus provides a plant, such as a soybean (Glycine max), which is resistant to a pathogen of viral, bacterial, fungal or oomycete origin, characterized in that the plant has a reduced level, reduced activity or complete absence of the DMR6 protein as compared to a plant that is not resistant to the said pathogen.

This form of resistance is in particular effective against pathogens of the phylum Oomycota, such as Albugo, Aphanomyces, Basidiophora, Bremia, Hyaloperonospora, Pachymetra, Paraperonospora, Perofascia, Peronophythora, Peronospora, Peronosclerospora, Phytium, Phytophthora, Plasmopara, Protobremia, Pseudoperonospora, Sclerospora, Viennotia species, as well as to pathogens belonging to the Fungi.

The resistance according to the present disclosure is based on an altered, in particular a reduced level, reduced activity or complete absence of the DMR6 protein in planta. The term “DMR6 protein” in this respect relates to the DMR6 gene product, such as the protein encoded by the At5g24530 gene in Arabidopsis. Such alterations can be achieved in various ways.

In one embodiment of the present disclosure, the reduced level of DMR6 protein is the result of a reduced endogenous DMR6 gene expression. Reducing the expression of the DMR6 gene can be achieved, either directly, e.g., by targeting DMR6, or indirectly by modifying the regulatory sequences thereof, or by stimulating repression of the gene. In some embodiments, endogenous DMR6 gene expression may be reduced by any suitable methodology including, without limitation, gene silencing, RNA interference (RNAi), virus-induced gene silencing

(VIGS), small RNA-mediated post-transcriptional gene silencing, transcription activator-like effector nuclease (TALEN) gene editing techniques, clustered Regularly Interspaced Short Palindromic Repeat (CRISPR/Cas9) gene editing techniques, and zinc-finger nuclease (ZFN) gene editing techniques.

Modulating the DMR6 gene to lower its activity or expression can be achieved at various levels. First, the endogenous gene can be directly mutated. This can be achieved by means of a mutagenic treatment. Alternatively, a modified DMR6 gene can be brought into the plant by means of transgenic techniques or by introgression, or the expression of DMR6 can be reduced at the regulatory level, for example by modifying the regulatory sequences or by modulating gene expression by, for example, gene silencing, RNA interference (RNAi), virus-induced gene silencing (VIGS), small RNA-mediated post-transcriptional gene silencing, transcription activator-like effector nuclease (TALEN) gene editing techniques, clustered Regularly Interspaced Short Palindromic Repeat (CRISPR/Cas9) gene editing techniques, or zinc-finger nuclease (ZFN) gene editing techniques.

In another embodiment of the present disclosure, the reduced level of DMR6 protein is the result of a mutation in the DMR6 gene resulting in a reduced DMR6 expression as compared to the wild-type DMR6 gene wherein no such mutation is present, or resulting in a reduced mRNA or protein stability. In a particular embodiment this is achieved by mutations in the DMR6 coding sequence that result in a non-functional DMR6 protein, i.e., without or with reduced enzymatic activity.

In another embodiment of the present disclosure, reduced expression can be achieved by down-regulation of DMR6 gene expression either at the transcriptional or the translational level, or by mutations that affect the expression of the DMR6 gene. Down-regulation of DMR6 gene expression can achieved by any suitable method, including without limitation, gene silencing, RNA interference (RNAi), virus-induced gene silencing (VIGS), small RNA-mediated post-transcriptional gene silencing, transcription activator-like effector nuclease (TALEN) gene editing techniques, clustered Regularly Interspaced Short Palindromic Repeat (CRISPR/Cas9) gene editing techniques, and zinc-finger nuclease (ZFN) gene editing techniques.

The present disclosure is based, at least in part, on research performed on resistance to Hyaloperonospora parasitica in Arabidopsis but is a general concept that can be more generally applied in plants, in particular in crop plants that are susceptible to infections with pathogens, such as Oomycota and Fungi.

The present disclosure is suitable for a large number of plant diseases caused by oomycetes such as, but not limited to, Phytophthora sojae on soybean, Bremia lactucae on lettuce, Peronospora farinosa on spinach, Pseudoperonospora cubensis on members of the Cucurbitaceae family, e.g., cucumber and melon, Peronospora destructor on onion, Hyaloperonospora parasitica on members of the Brassicaceae family, e.g., cabbage, Plasmopara viticola on grape, and Phytophthora infestans on tomato and potato.

When the modification of DMR6 gene expression in a plant is to be achieved via genetic modification of the DMR6 gene or via the identification of mutations in the DMR6 gene, and the gene is not yet known it must first be identified. To generate pathogen-resistant plants, in particular crop plants, via genetic modification of the DMR6 gene or via the identification of mutations in the DMR6 gene, the orthologous DMR6 genes must be isolated from these plant species.

Various methods are available for the identification of orthologous sequences in other plants.

A method for the identification of DMR6 orthologous sequences in a plant species, may for example comprise identification of DMR6 ESTs of the plant species in a database; designing primers for amplification of the complete DMR6 transcript or cDNA; performing amplification experiments with the primers to obtain the corresponding complete transcript or cDNA; and determining the nucleotide sequence of the transcript or cDNA. Suitable methods for amplifying the complete transcript or cDNA in situations where only part of the coding sequence is known are the advanced PCR techniques 5′RACE, 3′RACE, TAIL-PCR, RLM-RACE and vectorette PCR.

Alternatively, if no nucleotide sequences are available for the plant species of interest, primers are designed on the DMR6 gene of a plant species closely related to the plant of interest, based on conserved domains as determined by multiple nucleotide sequence alignment, and used to PCR amplify the orthologous sequence. Such primers are suitably degenerate primers.

Another reliable method to assess a given sequence as being a DMR6 ortholog is by identification of the reciprocal best hit. A candidate orthologous DMR6 sequence of a given plant species is identified as the best hit from DNA databases when searching with the Arabidopsis DMR6 protein or DNA sequence, or that of another plant species, using a Blast program. The obtained candidate orthologous nucleotide sequence of the given plant species is used to search for homology to all Arabidopsis proteins present in the DNA databases (e.g., at NCBI or TAIR) using the BlastX search method. If the best hit and score is to the Arabidopsis DMR6 protein, the given DNA sequence can be described as being an ortholog, or orthologous sequence.

DMR6 is encoded by a single gene in Arabidopsis as deduced from the complete genome sequence that is publicly available. In the genome of rice 3 orthologs, and in poplar 2 orthologs have been identified. In most other plant species tested so far, DMR6 appears to be encoded by a single gene, as determined by the analysis of mRNA sequences and EST data from public DNA databases. The orthologous genes and proteins are identified in these plants by nucleotide and amino acid comparisons with the information that is present in public databases.

Alternatively, if no DNA sequences are available for the desired plant species, orthologous sequences are isolated by heterologous hybridization using DNA probes of the DMR6 gene of Arabidopsis or another plant or by PCR methods, making use of conserved domains in the DMR6 coding sequence to define the primers. For many crop species, partial DMR6 mRNA sequences are available that can be used to design primers to subsequently PCR amplify the complete mRNA or genomic sequences for DNA sequence analysis.

In a specific embodiment the ortholog is a gene of which the encoded protein shows at least 50% identity with the Arabidopsis DMR6 protein (At5g24530) or that of other plant DMR6 proteins. In a more specific embodiment the identity is at least 55%, more specifically 60%, even more specifically 65%, 70%, 75%, 80%, 85%, 90%, 95% or 99%.

Accordingly, certain aspects of the present disclosure relate to a Phytophthora sojae resistant soybean plant, where the resistant soybean plant has a reduced activity of a DMR6 protein having amino acid sequence SEQ ID NO:115 and where the activity of the DMR6 protein is reduced in the resistant soybean plant compared to the activity of the DMR6 protein in a soybean plant that is not resistant to Phytophthoa sojae. Other aspects of the present disclosure relate to a

Phytophthora sojae resistant soybean plant, where the resistant soybean plant has a reduced activity of a DMR6 protein having amino acid sequence SEQ ID NO:116 and where the activity of the DMR6 protein is reduced in the resistant soybean plant compared to the activity of the DMR6 protein in a soybean plant that is not resistant to Phytophthoa sojae. Other aspects of the present disclosure relate to a Phytophthora sojae resistant soybean plant, where the resistant soybean plant has a reduced activity of a first DMR6 protein having amino acid sequence SEQ ID NO:115 and a reduced activity of a second DMR6 protein having amino acid sequence SEQ ID NO:116, and where the activity of the first DMR6 protein and the activity of the second DMR6 protein are reduced in the resistant soybean plant compared to the activity of the first DMR6 protein and the activity of the DMR6 second protein in a soybean plant that is not resistant to Phytophthora sojae.

In some embodiments, the resistant soybean plant has a non-natural mutation introduced into its genome that results in reduced expression or reduced transcription of a gene encoding the DMR6 protein having amino acid sequence SEQ ID NO:115. In some embodiments, the resistant soybean plant has a non-natural mutation introduced into its genome that results in reduced expression or reduced transcription of a gene encoding the DMR6 protein having amino acid sequence SEQ ID NO:116. In some embodiments, the resistant soybean plant has a non-natural mutation introduced into its genome that results in reduced expression or reduced transcription of the gene encoding the DMR6 protein having amino acid sequence SEQ ID NO:115 and a non-natural mutation introduced into its genome that results in reduced expression or reduced transcription of the gene encoding the DMR6 protein having amino acid sequence SEQ ID NO:116. In some embodiments, the non-natural mutations are achieved by, for example, gene silencing, RNA interference (RNAi), virus-induced gene silencing (VIGS), small RNA-mediated post-transcriptional gene silencing, transcription activator-like effector nuclease (TALEN) gene editing techniques, clustered Regularly Interspaced Short Palindromic Repeat (CRISPR/Cas9) gene editing techniques, and/or zinc-finger nuclease (ZFN) gene editing techniques.

Other aspects of the present disclosure relate to a seed, tissue, or plant part of the soybean plant of any of the preceding embodiments, where the seed, tissue, or plant part contains: reduced activity of the DMR6 protein having amino acid sequence SEQ ID NO:115; reduced activity of the DMR6 protein having amino acid sequence SEQ ID NO:116; or reduced activity of the DMR6 protein having amino acid sequence SEQ ID NO:115 and a reduced activity of the DMR6 protein having amino acid sequence SEQ ID NO:116.

Other aspects of the present disclosure relate to a method for obtaining a Phytophthora sojae resistant soybean plant by: reducing activity of a DMR6 protein having amino acid sequence SEQ ID NO:115 in a soybean plant. Other aspects of the present disclosure relate to a method for obtaining a Phytophthora sojae resistant soybean plant by: reducing activity of a DMR6 protein having amino acid sequence SEQ ID NO:116 in a soybean plant. Other aspects of the present disclosure relate to a method for obtaining a Phytophthora sojae resistant soybean plant by: reducing activity of a DMR6 protein having amino acid sequence SEQ ID NO:115 and reducing activity of a DMR6 protein having amino acid sequence SEQ ID NO:116 in a soybean plant. In some embodiments, reducing activity of the DMR6 protein having amino acid sequence SEQ ID NO:115 is achieved in the soybean plant by introducing a non-natural mutation into its genome that results in reduced expression or reduced transcription of a gene encoding the DMR6 protein having amino acid sequence SEQ ID NO:115. In some embodiments, reducing activity of the DMR6 protein having amino acid sequence SEQ ID NO:116 is achieved in the soybean plant by introducing a non-natural mutation into its genome that results in reduced expression or reduced transcription of a gene encoding the DMR6 protein having amino acid sequence SEQ ID NO:116.

In some embodiments, reducing activity of the DMR6 protein having amino acid sequence SEQ ID

NO:115 is achieved in the soybean plant by introducing a non-natural mutation into its genome that results in reduced expression or reduced transcription of a gene encoding the DMR6 protein having amino acid sequence SEQ ID NO:115, and where reducing activity of the DMR6 protein having amino acid sequence SEQ ID NO:116 is achieved in the soybean plant by introducing a non-natural mutation into its genome that results in reduced expression or reduced transcription of a gene encoding the DMR6 protein having amino acid sequence SEQ ID NO:116. In some embodiments, the non-natural mutations are achieved by, for example, gene silencing, RNA interference (RNAi), virus-induced gene silencing (VIGS), small RNA-mediated post-transcriptional gene silencing, transcription activator-like effector nuclease (TALEN) gene editing techniques, clustered Regularly Interspaced Short Palindromic Repeat (CRISPR/Cas9) gene editing techniques, and zinc-finger nuclease (ZFN) gene editing techniques.

Other aspects of the present disclosure relate to a Phytophthora sojae resistant soybean plant produced by the method of any of the preceding embodiments, where the soybean plant has reduced activity of the DMR6 protein having amino acid sequence SEQ ID NO:115. Other aspects of the present disclosure relate to a Phytophthora sojae resistant soybean plant produced by the method of any of the preceding embodiments, where the soybean plant has reduced activity of the DMR6 protein having amino acid sequence SEQ ID NO:116. Other aspects of the present disclosure relate to a Phytophthora sojae resistant soybean plant produced by the method of any of the preceding embodiments, where the soybean plant has reduced activity of the DMR6 protein having amino acid sequence SEQ ID NO:115 and a reduced activity of the DMR6 protein having amino acid sequence SEQ ID NO:116. Other aspects of the present disclosure relate to a seed, tissue, or plant part of the resistant soybean plant of any of the preceding embodiments, where the seed, tissue, or plant part has reduced activity of the DMR6 protein having amino acid sequence SEQ ID NO:115. Other aspects of the present disclosure relate to a seed, tissue, or plant part of the resistant soybean plant of any of the preceding embodiments, where the seed, tissue, or plant part has reduced activity of the DMR6 protein having amino acid sequence SEQ ID NO:116. Other aspects of the present disclosure relate to a seed, tissue, or plant part of the resistant soybean plant of any of the preceding embodiments, where the seed, tissue, or plant part has reduced activity of the DMR6 protein having amino acid sequence SEQ ID NO:115 and a reduced activity of the DMR6 protein having amino acid sequence SEQ ID NO:116.

### BRIEF DESCRIPTION OF THE DRAWINGS

FIG. 1A-1D show the alignment of the amino acid sequences of the DMR6 protein of Arabidopsis thaliana (SEQ ID NO:62) and orthologs from Aquilegia species (SEQ ID NO:63), Citrus sinensis (SEQ ID NO:64), Coffea canephora (SEQ ID NO:65), Cucumis sativus (SEQ ID NO:67), Gossypium hirsutum (SEQ ID NO:68), Lactuca sativa (SEQ ID NO:70), Medicago truncatula (SEQ ID NO:71), Oryza sativa (SEQ ID NOs. 72-74), Populus trichocarpa (SEQ ID NOs. 75 and 76), Solanum lycopersicum (SEQ ID NOs. 77 and 78), Sorghum bicolor (SEQ ID NO:79), Spinacia oleracea (SEQ ID NO:81), Vitis vinifera (SEQ ID NO:82), Zea mays (SEQ ID NO:83), and Zingiber officinale (SEQ ID NO:84), using the CLUSTAL W (1.83) multiple sequence alignment programme (EBI). Below the sequences the conserved amino acids are indicated by the dots, and the identical amino acids are indicated by the asterisk.

FIG. 2 shows the nucleotide (SEQ ID NO:61) and amino acid sequence (SEQ ID NO:62) of the DMR6 gene (At5g24530, gi 42568064, Genbank NM_122361) and protein (gi 15238567, Genbank NP_197841) of Arabidopsis thaliana, respectively.

FIG. 3 shows the nucleotide (SEQ ID NO:69) and derived amino acid sequence (SEQ ID NO:70) of the DMR6 ortholog of Lactuca sativa, respectively.

FIG. 4 shows the nucleotide (SEQ ID NO:80) and derived amino acid sequence (SEQ ID NO:81) of the DMR6 ortholog of Spinacia oleracea, respectively.

FIG. 5 shows the nucleotide (SEQ ID NO:66) and derived amino acid sequence (SEQ ID NO:67) of the DMR6 ortholog of Cucumis sativus and Cucumis melo.

FIG. 6A and 6B show the downy mildew resistance of the Arabidopsis dmr6 mutants.

FIG. 6A shows quantification of sporangiophores of H. parasitica isolate Waco9, 7 days post inoculation, on the dmr6-1 mutant (BC₂, line E37) compared to its parental line Ler eds1-2 and on the dmr6-2 mutant (FLAG_445D09 T-DNA line) compared to its parental line Ws-4. FIG. 6B shows restoration of susceptibility by complementation with the At5g24530 gene in the dmr6-1 mutant. H. parasitica spores per mg seedling weight were quantified on Ler eds1-2, dmr6-1 and 5 complementation lines (#121, 122, 211,231, and 241).

FIG. 7 shows the structure of the Arabidopsis DMR6 gene and dmr6-1 and dmr6-2 mutations. The DMR6 gene contains four exons and a coding sequence of 1026 bases. The two alleles are indicated; dmr6-1 with a base change in exon 2, and dmr6-2 with a T-DNA insertion into intron 2.

FIG. 8 shows the relative transcript levels of DMR6 in Ler plants either mock treated or inoculated with a compatible or incompatible H. parasitica isolate. Transcript levels were determined at different days post inoculation. The difference in cycle threshold (ACT) values reflect the number of additional PCR amplification cycles required to reach an arbitrary threshold product concentration as compared to ACTIN2. A lower ACT value indicates a higher transcript level.

FIG. 9A-9E show the expression of the DMR6 promoter-reporter (pDMR6::GUS) construct in transgenic Arabidopsis lines, visualized with only X-gluc as substrate (FIG. 9D and 9E) or Magenta-Xgluc (FIG. 9A-9C) and trypan blue staining of H. parasitica growth. FIG. 9A shows Ler eds1-2 (pDMR6::GUS) 3dpi with H. parasitica, Cala2 isolate. FIG. 9B shows Col-0 (pDMR6::GUS) 3dpi with H. parasitica, Waco9 isolate. FIG. 9C shows Ler eds1-2 (pDMR6::GUS) 3dpi with H. prasitica, Emoy2 isolate. FIG. 9D shows Col-0 (pDMR6::GUS) 3 dp wounding. FIG. 9E shows Col-0 (pDMR6::GUS) 3 dp BTH application.

FIG. 10A-B shows the Q-PCR analysis of the transcript levels of the genes; At4g14365, At1g14880, ACD6, PR-1, PR-2 and PR-5, selected as up regulated in the dmr6-1 micro array analysis. FIG. 10A shows transcription levels of the six genes in dmr6-1 compared to Ler eds1-2 and additionally the DMR6 transcript. FIG. 10B shows elevated gene transcripts of six defense-associated genes in dmr6-2 versus Ws-4. ACT reflects the number of additional PCR amplification cycles required to reach the level of ACTIN2 transcripts. A lower ACT value indicates a higher transcript level.

FIG. 11 shows the nucleotide sequence (SEQ ID NO:107) of the 3 kb region upstream of the start codon of the DMR6 gene (at5g24530) of Arabidopsis thaliana, including the promoter and 5′ -UTR (underlined).

FIG. 12 shows the nucleotide (SEQ ID NO:95) and derived amino acid sequence (SEQ ID NO:96) of the DMR6 ortholog of Solanum lycopersicum, respectively.

FIG. 13 shows the nucleotide (SEQ ID NO:97) and derived amino acid sequence (SEQ ID NO:98) of the DMR6 ortholog of Nicotiana benthamiana, respectively.

FIG. 14 shows complementation of Arabidopsis thaliana dmr6-1 with DMR6 derived from Cucumis sativa (Cs), Spinacia oleracea (Si), Lactuca sativa (Ls) and Solanum lycopersicum (So).

### DETAILED DESCRIPTION

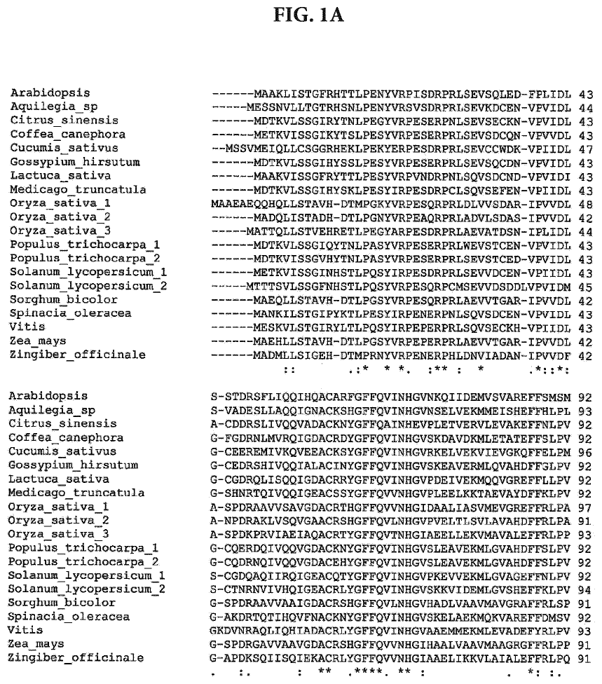

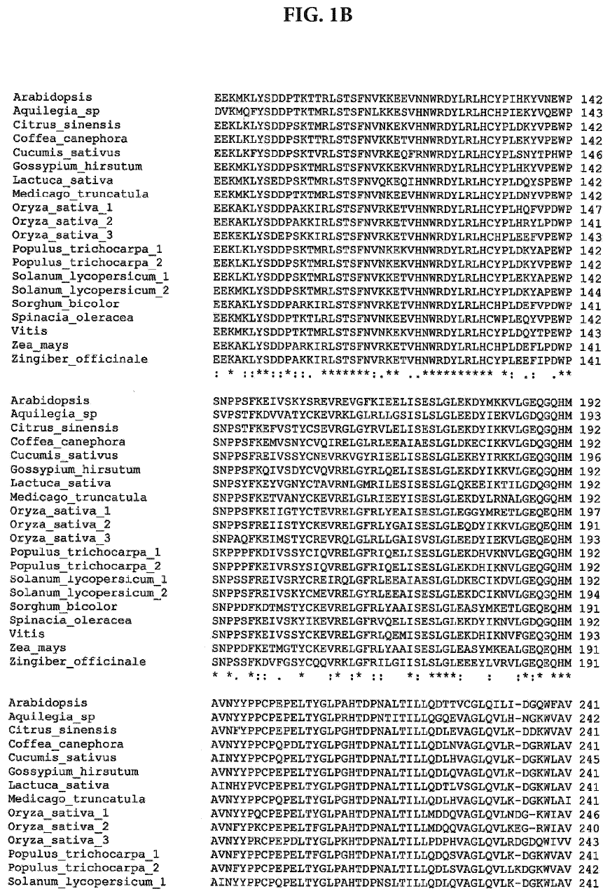

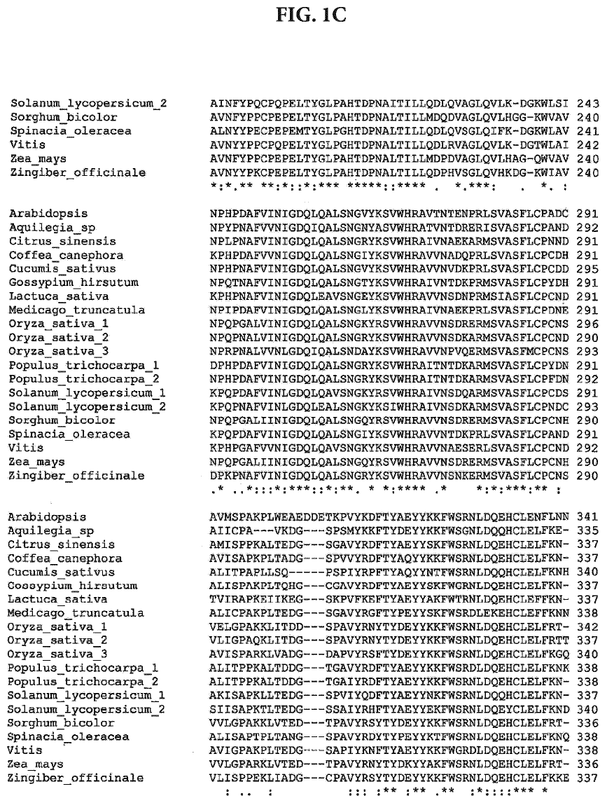

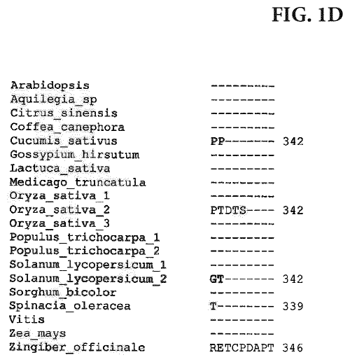

FIG. 1 shows orthologous DMR6 sequences (described in Table 1) that have been identified in publicly available databases and obtained by PCR amplification on cDNA and subsequent sequencing. After orthologous DMR6 sequences are identified, the complete nucleotide sequence of the regulatory and coding sequence of the gene is identified by standard molecular biological techniques. For this, genomic libraries of the plant species are screened by DNA hybridization or PCR with probes or primers derived from a known DMR6 gene to identify the genomic clones containing the DMR6 gene. Alternatively, advanced PCR methods, such as RNA ligase-mediated RACE (RLM-RACE), can be used to directly amplify gene and cDNA sequences from genomic DNA or reverse-transcribed mRNA. DNA sequencing subsequently results in the characterization of the complete gene or coding sequence.

Once the DNA sequence of the gene is known this information is used to prepare the means to modulate the expression of the DMR6 gene.

To achieve a reduced DMR6 protein level, the expression of the DMR6 gene can be down-regulated or the enzymatic activity of the DMR6 protein can be reduced by amino acid substitutions resulting from nucleotide changes in the DMR6 coding sequence.

In a particular embodiment, downregulation of DMR6 gene expression is achieved by gene silencing, RNA interference (RNAi), virus-induced gene silencing (VIGS), small RNA-mediated post-transcriptional gene silencing, transcription activator-like effector nuclease (TALEN) gene editing techniques, clustered Regularly Interspaced Short Palindromic Repeat (CRISPR/Cas9) gene editing techniques, and/or zinc-finger nuclease (ZFN) gene editing techniques. For this, transgenic plants are generated expressing one or more constructs targeting DMR6. These constructs may include, without limitation, an anti-sense construct, an optimized small-RNA construct, an inverted repeat construct, a targeting construct, a guide RNA construct, a construct encoding a targeting protein, and/or a combined sense-anti-sense construct, and may work in conjunction with a nuclease, an endonuclease, and/or an enzyme, so as to downregulate DMR6 gene expression

In an alternative embodiment, one or more regulators of the DMR6 gene are downregulated (in case of transcriptional activators) by RNA interference (RNAi), virus-induced gene silencing (VIGS), small RNA-mediated post-transcriptional gene silencing, transcription activator-like effector nuclease (TALEN) gene editing techniques, clustered Regularly Interspaced Short Palindromic Repeat (CRISPR/Cas9) gene editing techniques, and/or zinc-finger nuclease (ZFN) gene editing techniques.

In another embodiment regulators are upregulated (in case of repressor proteins) by transgenic overexpression. Overexpression is achieved in a particular embodiment by expressing repressor proteins of the DMR6 gene from a strong promoter, e.g., the 35S promoter that is commonly used in plant biotechnology.

The downregulation of the DMR6 gene can also be achieved by mutagenesis of the regulatory elements in the promoter, terminator region, or potential introns. Mutations in the DMR6 coding sequence in many cases leads to amino acid substitutions or premature stop codons that negatively affect the expression or activity of the encoded DMR6 protein.

These mutations are induced in plants by using mutagenic chemicals such as ethyl methane sulfonate (EMS), by irradiation of plant material with gamma rays or fast neutrons, or by other means. The resulting nucleotide changes are random, but in a large collection of mutagenized plants the mutations in the DMR6 gene can be readily identified by using the TILLING (Targeting Induced Local Lesions IN Genomes) method (McCallum et al. (2000) Targeted screening for induced mutations. Nat. Biotechnol. 18, 455-457, and Henikoff et al. (2004) TILLING. Traditional mutagenesis meets functional genomics. Plant Physiol. 135, 630-636). The principle of this method is based on the PCR amplification of the gene of interest from genomic DNA of a large collection of mutagenized plants in the M2 generation. By DNA sequencing or by looking for point mutations using a single-strand specific nuclease, such as the CEL-I nuclease (Till et al. (2004) Mismatch cleavage by single-strand specific nucleases. Nucleic Acids Res. 32, 2632-2641) the individual plants that have a mutation in the gene of interest are identified.

By screening many plants, a large collection of mutant alleles is obtained, each giving a different effect on gene expression or enzyme activity. The gene expression or protein levels can for example be tested by analysis of DMR6 transcript levels (e.g., by RT-PCR) or by quantification of DMR6 protein levels with antibodies.

Plants with the desired reduced DMR6 level or DMR6 expression are then back-crossed or crossed to other breeding lines to transfer only the desired new allele into the background of the crop wanted.

The present disclosure further relates to mutated DMR6 genes. In a particular embodiment, the present disclosure relates to dmr6 alleles with premature stop codons, such as the dmr6-1 allele.

In another embodiment, the present disclosure relates to mutated versions of the

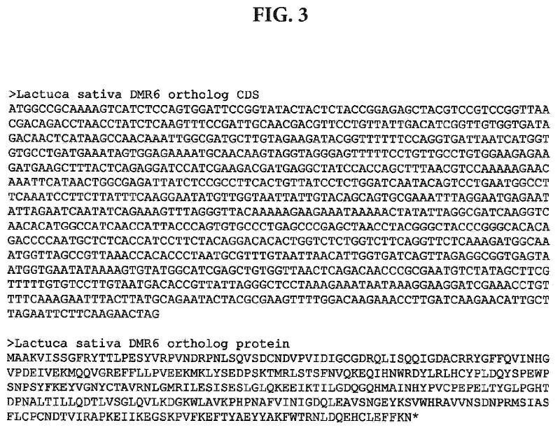

DMR6 genes of Lactuca sativa, Cucumis sativus, and Spinacia oleracea as shown in FIG. 3-5.

The present disclosure demonstrates that plants having no or a reduced level of functional DMR6 gene product show resistance to pathogens, in particular of oomycete and fungal origin. With such knowledge the skilled person can identify so far unknown natural variants of a given plant species that have variants of the DMR6 gene that lead to a reduced level or absence of a functional DMR6 protein, or mutated versions of the DMR6 protein, and to use these natural variants according to the present disclosure.

The present disclosure further relates to the use of a DMR6 promotor for providing disease resistance into plants, i.e., for providing plants with a resistance to a pathogen of viral, bacterial, fungal or oomycete origin. According to the present disclosure, the transcriptional up-regulation of DMR6 in response to pathogen infection has been demonstrated. Both transcript analysis as well as promotor DMR6-reporter lines support this finding (see Example 1, below). The pathogen-inducible DMR6 promotor according to the present disclosure thus is particularly useful to control the expression of inducible systems that lead to disease resistance in plants.

One example of such inducible system that leads to disease resistance in plants, and in which the DMR6 promotor according to the present disclosure may be effective, has e.g., been described in WO 99/45125, wherein an antisense nucleotide sequence for a gene involved in the regulation of the C-5 porphyrin metabolic pathway is operably linked to a pathogen-inducible promotor and used to transform plant cells. Expression of the antisense nucleotide sequence in response to the pathogen effectively disrupts porphyrin metabolism of the transformed plant cell, and development of a localized lesion wherein the spread of the pathogen is contained. WO 96/36697 also discloses inducible systems leading to disease resistance in plants, wherein an inducible promotor controls the expression of a protein capable of evoking the hypersensitivity response in a plant. EP 0474857 furthermore discloses a method for the induction of pathogen resistance in plants, comprising transforming plants with polynucleotide sequences encoding a pair of pathogen-derived-avirulence-gene/plant-derived-resistance gene, wherein the expression of one of or both the elicitor peptide and the resistance gene is regulated by a pathogen inducible promotor. Further examples of inducible systems leading to resistance to pathogens in plants have been described in e.g., WO 98/32325.

In a particular preferred embodiment, the present disclosure relates to a method of providing disease resistance in a plant, comprising transforming a plant cell with a DNA construct comprising at least one expressible nucleic acid which is operably linked to a pathogen-inducible promotor that is operable within a plant cell, and regenerating transformed plants from said plant cells, wherein the pathogen-inducible promotor is a DMR6 promotor, and wherein the expression of the expressible nucleic acid confers disease resistance to the transgenic plant.

The present disclosure also relates to disease resistance plants, obtainable by said method, as well as to plant tissue, and seeds obtained from said plants.

The present disclosure in particular relates to plants, which are resistant to a pathogen of viral, bacterial, fungal or oomycete origin, wherein the plant comprises in its genome a DNA construct, comprising at least one expressible nucleic acid which is operably linked to a pathogen-inducible promotor, wherein the pathogen-inducible promotor is a DMR6 promotor.

The present disclosure also relates to the DNA construct per se, comprising at least one expressible nucleic acid which is operably linked to a pathogen-inducible promotor, wherein the pathogen-inducible promotor is a DMR6 promotor. The construct of the present disclosure can be used to transform plant cells which may be regenerated into transformed plants. Furthermore, transformed plant tissue and seed may be obtained. Suitable methods for introducing the construct of the present disclosure into plant cells are known to the skilled person.

As used herein, “operably linked” refers to a promotor and an expressible nucleic acid, e.g., a gene, are connected in such way as to permit initiation of transcription of the expressible nucleic acid (e.g., gene) by the promotor.

By “expressible nucleic acid” is meant a nucleic acid (e.g., a gene, or part of a gene) that can be expressed in the cell, i.e., that can be transcribed into mRNA, and eventually may be translated into a protein. The expressible nucleic acid may be genomic DNA, cDNA, or chemically synthesized DNA or any combination thereof.

According to the present disclosure, a DNA construct comprises all necessary nucleic acid elements which permit expression (i.e., transcription) of a particular nucleic acid in a cell. Typically, the construct includes an expressible nucleic acid, i.e., a nucleic acid to be transcribed, and a promotor. The construct can suitably be incorporated into e.g., a plasmid or vector.

The expressible nucleic acid preferably is a gene involved in a plant defense response, e.g., a gene associated with the hypersensitivity response of a plant. In the hypersensitivity response (HR) of a plant, the site in the plant where the pathogen invades undergoes localized cell death by the induced expression of a suicide mechanism that triggers said localized cell death in response to pathogens. In this way, only a few plant cells are sacrificed and the spread of the pathogen is effectively arrested. Examples of said genes involved in a plant defense response are the regulatory protein NPR1/NIM1 (Friedrich et al., Mol. Plant Microbe Interact. 14(9): 1114-1124, 2001) and the transcription factor MYB30 (Vailleau et al., Proc. Natl. Acad. Sci. USA 99(15): 10179-10184, 2002).

In a particular embodiment, the expressible nucleic acid encodes an autologous or heterologous polypeptide capable of conferring disease-resistance to a plant. By “autologous polypeptide” is meant any polypeptide that is expressed in a transformed plant cell from a gene that naturally occurs in the transformed plant cell. By “heterologous polypeptide” is meant any polypeptide that is expressed in a transformed plant cell from a gene that is partly or entirely foreign (i.e., does not naturally occur in) to the transformed plant cell. Examples of such polypeptides are the mammalian Bax protein, which encodes a pro-apoptotic protein and results in cell death in plants (Lacomme and Santa Cruz, Proc. Natl. Acad. Sci. USA 96(14): 7956-61, 1999) and fungal chitinases (de las Mercedes Dana et al., Plant Physiol. 142(2): 722-730, 2006).

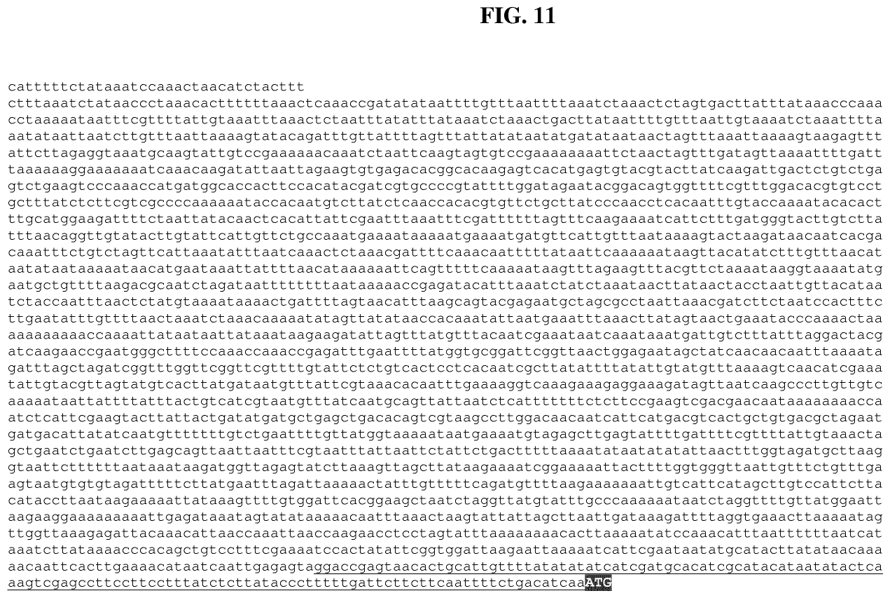

Preferably, the DMR6 promotor is the Arabidopsis DMR6 promotor. The DMR6 promotor comprises a region of 3000 bp that is upstream of the Arabidopsis DMR6 coding sequence (ATG start codon) and includes the 5′UTR. Preferably the DMR6 promotor comprises a nucleotide sequence as defined in FIG. 11, and/or any functional fragment thereof, i.e., any fragment (or part) of said sequence which still is capable of initiating transcription of the expressible nucleic acid(s) to which it is operably linked, and/or natural variants thereof, i.e., natural variants of this promotor which may contain small polymorphisms, but which are generally at least 90% identical.

In a further embodiment, the DMR6 promotor is an orthologous DMR6 promotor, i.e., a promotor of an orthologous DMR6 gene. Methods for identifying DMR6 orthologs have been described in Example 2 below. Once the DMR6 orthologs have been identified, the skilled person will be able to isolate the respective promotor of said orthologs, using standard molecular biological techniques.

According to the present disclosure, the DMR6 promotor has been shown to be strongly pathogen-induced, and the DMR6 promotor is not highly expressed in other non-infected tissues. Thus, it is a very suitable promotor for use in inducible systems for providing resistance to pathogens of viral, bacterial, fungal or oomycete origin in plants. Examples of specific pathogens and plants for which the inducible system, using the DMR6 promotor of the present disclosure, suitably can be used, have been given above.

The present disclosure is illustrated in the following examples that are not intended to limit the present disclosure in any way. In the examples reference is made to the figures described above and the following tables.

Table 1 shows the Genbank accession numbers and GenInfo identifiers of the Arabidopsis DMR6 mRNA and orthologous sequences from other plant species.

Table 2 shows the PCR primers for the markers used for the map-based cloning of DMR6.

Table 3 shows primer pairs for cloning dmr6 orthologs in a suitable plant expression vector.

The present disclosure will be more fully understood by reference to the following Examples. They should not, however, be construed as limiting the scope of the present disclosure.

### EXAMPLES

### Example 1

### The Arabidopsis DMR6 (At5g24530) Gene is Required for Downy Mildew Susceptibility

### Experimental Procedures

### Hyaloperonospora Parasitica Growth and Infection

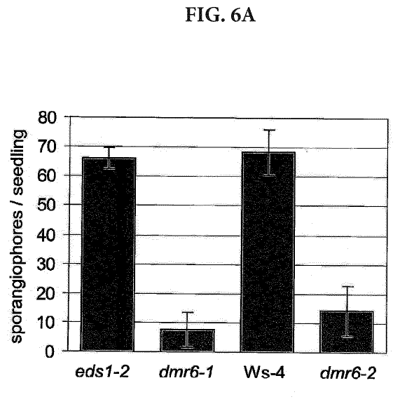

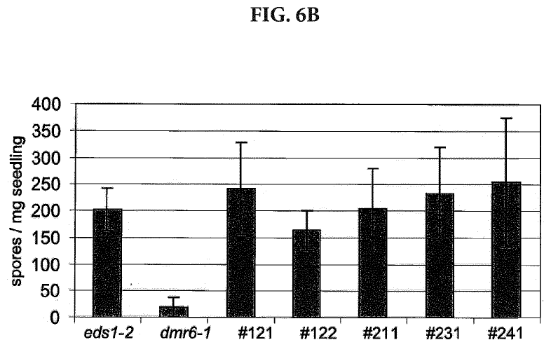

H. parasitica isolate Waco9 was provided by Dr. M. Aarts (WUR, Wageningen, NL) and isolate Cala2 provided by Dr. E. Holub (Warwick HRI, Wellsbourne, UK) and maintained on Arabidopsis Ws-0 and Ler, respectively. Inocula (400,000 spores per ml) were weekly transferred to 10 day old healthy seedlings (Holub, E. B. et al., Mol. Plant Microbe Interact. 7: 223-239, 1994) by use of a spray gun. Seedlings were air-dried for approximately 45 minutes and incubated under a sealed lid at 100% relative humidity in a growth chamber at 16° C. with 9 hours of light per day (100mE/m2/s). The sporulation levels were quantified 7 days post inoculation (dpi) by counting the number of sporangiophores per seedling, for at least 40 seedlings per tested line (FIG. 6A) or by isolating spores in water 5 dpi and determining the spore concentration to give the number per mg leaf tissue (FIG. 6B).

Generation of Backcrossed dmr6 Lines

The dmr6 mutants were back crossed twice (BC₂) to the parental line Ler eds1-2 as well as Ler. The BC₂ lines generated with Ler were selected for the presence of the wild type EDS1 gene by PCR analysis.

### Cloning DMR6

Fine mapping of the dmr6 gene was done with PCR markers designed using the Cereon database to identify insertion and deletion (IND) differences between Col-0 and Ler. The markers: IND_MOP9 in gene At5G24210; IND_K16H17 in gene At5G24420; IND_T4C12 in gene At5G24820; IND_T11H3 in between genes At5G24950_60 and IND_F21J6 in gene At5G25270 were used for mapping (Table 2). An additional screen for new recombinants was initiated on 300 F₂ plants resulting in eight F₂ recombinant plants between the two IND based markers IND_MOP9 and IND_T4C12, which flanked a region of 61 genes. Seven additional markers (M450-M590; Table 2) reduced the region to eighteen candidate genes for the dmr6 locus, between At5g24420 and At5g24590. Sequence analysis of At5g24530 indicated a point mutation leading to a stop codon in exon 2 in the dmr6-1 mutant.

Identification of a dmr6 T-DNA Insertion Line

A second dmr6 allele was identified, 445D09 a FLAG T-DNA insertion line generated by INRA Versailles in the Ws-4 accession background. The T-DNA insertion was confirmed by PCR using a primer designed in the At5g24530 gene, LP primer (5′-caggtttatggcatatctcacgtc-3′) (SEQ ID NO:108), in combination with the T-DNA right border primer, Tag3′ (5′-tgataccagacgttgcccgcataa-3′) (SEQ ID NO:109) or RB4 (5′-tcacgggttggggtttctacaggac-3′) (SEQ ID NO:110). The exact T-DNA insertion in the second intron of At5g24530 was confirmed by sequencing of amplicons generated with the T-DNA primers from both the left and right border in combination with the gene specific primers LP or RP (5′-atgtccaagtccaatagccacaag-3′) (SEQ ID NO:111).

cDNA Synthesis

RNA was isolated (from approximately 100 mg leaf tissue from 10 day old seedlings) with the RNaesy kit (Qiagen, Venlo, The Netherlands) and treated with the RNase-free DNase set (Qiagen). Total RNA was quantified using an UVmini-1240 spectrophotometer (Shimadzu, Kyoto, Japan). cDNA was synthesized with Superscript III reverse transcriptase (Invitrogen, Carlsbad, Calif., USA) and oligo (dT)15 (Promega, Madison, Wis., USA), according manufactures instructions.

Complementation of the dmr6-1 mutant

Complementation lines were generated by transforming dmr6 plants by the floral dip method with Agrobacterium tumefaciens (Clough and Bent, 1998) containing the At5g24530 gene from Col-0 behind the 35S promoter. The construct was generated by PCR amplification of the full length At5g24530 from Col-0 cDNA with primers which included restriction sites that were used for directional cloning. A forward primer (5′-ttctgggatccaATGGCGGCAAAGCTGATATC-3′)

(SEQ ID NO:1) containing a BamHI restriction site near the start codon (ATG), amplified the 5′-end of DMR6 and at the 3′-end after the stop codon an EcoRI site was generated with a reverse primer (5′-gatatatgaattcttagttgtttagaaaattctcgaggc-3′) (SEQ ID NO:2). The 355-DMR6-Tn was cloned into the pGreenII0229 (Hellens, R. P., Edwards, E. A., Leyland, N. R., Bean, S., and Mullineaux, P. M. (2000)). pGreen: a versatile and flexible binary Ti vector for Agrobacterium-mediated plant transformation. Plant Mol. Biol. 42, 819-832). 300 μM DL-Phosphinothricin (BASTA) resistant seedlings were isolated and analyzed for H. parasitica susceptibility and for DMR6 expression levels by RT-PCR.

### Knock Down Lines of DMR6 by RNAi

RNAi lines were generated in the Ler eds1-2 and Col-0 background. A 782 bp long cDNA amplicon of Col-0 At5g24530 gene was generated. The PCR was done with the Phusion DNA polymerase (2U/μL) and two different primer combinations. The amplicon from the first DMR6 gene specific primer combination

(SEQ ID NO: 3)
(RNAiDMR6F:
5′-aaaaagcaggctGACCGTCCACGTCTCTCTGAA-3′
and
(SEQ ID NO: 4)
RNAiDMR6R:
5′-AGAAAGCTGGGTGAAACGATGCGACCGATAGTC-3′)

was used as a template for the second PCR amplification with general primers allowing recombination into the pDONR7 vector of the GateWay cloning system. For the second PCR 10 μ1 of the first PCR (denaturation for 30 sec. at 98° C. followed by 10 cycles of: 10 sec. at 98° C.; 30 sec. at 58° C.; 30 sec. at 72° C.) in a total volume of 20 μl was used as template. The second PCR (denaturation for 30 sec. at 98° C. followed by 5 cycles of: 10 sec. at 98° C.; 30 sec. at 45 ° C.; 30 sec. at 72° C. and 20 cycles of 10 sec. at 98° C.; 30 sec. at 55° C.; 30 sec. at 72° C. finished by a final extension of 10 min. at 72° C.) with the attB1 (5′-GGGACAAGTTTGTACAAAAAAGCAGGCT-3′) (SEQ ID NO:5) and the

(SEQ ID NO: 6)
attB2 (5′-ggggaccactttgtacaagaaagctgggt-3′)

were performed in a 50 μl reaction volume. PCR product was gel purified and 50 ηg insert was recombined into 150 μl pDONR7 vector with the clonase BP enzyme. The vector was transformed into electrocompotent

DH5α E.coli cells and plasmids containing the correct insert were isolated and 100 rᵢg of the pDONR7 with the DMR6 amplicon were used in the LR reaction to recombine the insert in two opposite direction into 150 ηg pHellsgate8 vector. After transformation into E.coli, Spectomycin resistant clones were selected and the isolated plasmids were verified by a NotI digest for the right insert size and by colony PCR with a single internal primer for At5G24530 (DfragmentF: 5′-gagaagtgggatttaaaatagaggaa-3′) (SEQ ID NO:7), if the inserts was inserted twice in opposite direction an amplicon of 1420 bp could be detected. Correct pHellsgate8 plasmids with the double insert in opposite directions were transformed into electrocompotent Agrobacterium strain, C58C1. Plasmids were isolated from the Agrobacterium and retransformed into the E.coli to confirm the right size of the plasmid and the insert by NotI digestion. The reconfirmed Agrobacterium strains were used for the floral dip transformation of the Col-0 and Ler eds1-2 plants. The developed seeds were screened for Kanamycin resistance on ½× GM plates, the T₁ seedlings were transferred and the next generation of seeds the T₂ was analyzed for DMR6 expression and H. parasitica susceptibility.

Gene Expression Profiling of the dmr6 Mutant

Total RNA was isolated as described above. mRNA was amplified with the MessageAmp aRNA kit (Ambion). CATMA array (Crowe et al., 2003) slides containing approximately 25,000 gene specific tags were hybridized according to standardized conditions described by de Jong et al. (de Jong M., van Breukelen B., Wittink, F. R., Menke, F. L., Weisbeek, P. J., and Van den Ackerveken G. (2006). Membrane-associated transcripts in

Arabidopsis; their isolation and characterization by DNA microarray analysis and bioinformatics. Plant J. 46, 708-721). For quantitative PCR, cDNA templates were generated as described previously. Cycle thresholds were determined per transcript in triplicate using the ABI PRISM 7700 sequence detection system (Applied Biosystems, Foster City, Calif., USA) using SYBR Green I (Applied Biosystems, Foster City, Calif., USA) as reporter dye. Primer sets for the transcripts are:

DMR6
(SEQ ID NO: 8)
(QDMR6F:5′-TGTCATCAACATAGGTGACCAG-3′
and
(SEQ ID NO: 9)
QDMR6R:5′-CGATAGTCACGGATTTTCTGTG-3′),
At1g14880
(SEQ ID NO: 10)
(QAt1g14880F: 5′-CTCAAGGAGAATGGTCCACA-3′
and
(SEQ ID NO: 11)
QAt1g14880R:5′-CGACTTGGCCAAATGTGATA-3′),
At4g14365
(SEQ ID NO: 12)
(QAt4g14365F: 5′-TGGTTTTCTGAGGCATGTAAA-3′
and
(SEQ ID NO: 13)
QAt4g14365R:5′-AGTGCAGGAACATTGGTTGT-3′)
ACD6
(SEQ ID NO: 14)
(QACD6F:5′-TGGACAGTTCTGGAGCAGAT-3′,
and
(SEQ ID NO: 15)
QACD6R:5′-CAACTCCTCCGCTGTGAG-3′),
PR-5
(SEQ ID NO: 16)
(QPR-5F:5′-GGCAAATATCTCCAGTATTCACA-3′
and
(SEQ ID NO: 17)
QPR-5R:5′-GGTAGGGCAATTGTTCCTTAGA-3′),
PR-2
(SEQ ID NO: 18)
(QPR-2 F:5′-AAGGAGCTTAGCCTCACCAC-3′
and
(SEQ ID NO: 19)
QPR-2R:5′-GAGGGAAGCAAGAATGGAAC-3′),
PR-1
(SEQ ID NO: 20)
(QPR-1F:5′-GAACACGTGCAATGGAGTTT-3′
and
(SEQ ID NO: 21)
QPR-1R:5′-GGTTCCACCATTGTTACACCT-3′)
and
ACT-2
(SEQ ID NO: 22)
(QACT2 F:5′-AATCACAGCACTTGCACCA-3′
and
(SEQ ID NO: 23)
QACT2R:5′-GAGGGAAGCAAGAATGGAAC-3′)
generating 100 base pair fragments.

### Results

Characterization of the Gene Responsible for Pathogen Resistance in the dmr6 Mutant

Van Damme et al., 2005, supra disclose a dmr6 mutant that is resistant to H. parasitica. The level of resistance can be examined by counting the number of sporangiophores per seedling seven day post inoculation with the H. parasitica (isolate Waco9 or Cala2, obtainable from Dr. G. Van den Ackerveken, Plant-Microbe Interactions Group, University of Utrecht, Utrecht, NL). The parental line, Ler edsl-2 (Parker et al., 1996, Plant Cell 8:2033-2046), which is highly susceptible, is used as a positive control (and is set at 100%).

The reduction in sporangiophore formation on the infected dmr6 mutants compared to seedlings of the parental lines is shown in FIG. 6a, wherein the results of the quantification of Hyaloperonospora parasitica, Waco9 sporulation (sporangiophores/seedling) on the downy mildew resistant dmr6-1 mutant, back-crossed twice to the parental line Ler eds1-2, and on mutant dmr6-2 (FLAG_445D09 T-DNA line) compared to the control lines is shown.

According to the present disclosure, the gene responsible for resistance to H.

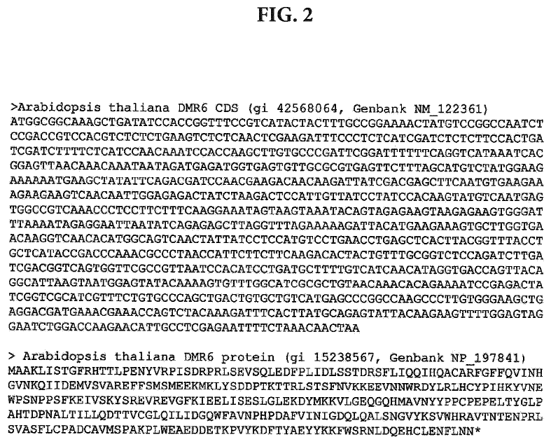

parasitica in the dmr6 mutants of van Damme et al., 2005, supra, has been cloned by a combination of mapping and sequencing of candidate genes. Previously, the recessive dmr6 mutation was mapped near the nga139 marker on chromosome 5 to a region encompassing 74 genes. Fine mapping linked the dmr6 locus to a mapping interval containing the BACs T13K7 and K18P6 between the markers At5g24420 and At5g24590 located in the corresponding genes. This allowed the dmr6 interval to be confined to a region of 18 candidate genes. Comparative sequence analysis of the 18 genes in dmr6 and the parental line, Ler eds1-2 revealed a point mutation in the second exon of the At5g24530 gene. This single base change of G to A, typical for an EMS mutation, changes a TGG a (trp codon) to a TGA (premature stop codon) at nucleotide position 691 of the coding sequence (FIG. 7). The early stop codon truncates the predicted oxidoreductase enzyme of 342 aa at position 141 before the conserved catalytic domain suggesting that dmr6 is a null-allele. The At5g24530 coding sequence (FIG. 2) is predicted to encode a protein with a mass of 39.4 kDa. No biological role has so far been described for At5g24530.

### At5g24530 is DMR6

A second allele, dmr6-2, was identified in a T-DNA insertion line (FLAG_445D09) from the mutant collection from INRA, Versailles. The presence and location of the T-DNA insert in the second intron of At5g24530 (FIG. 7) was confirmed by PCR and sequence analysis (data not shown). Progeny of the FLAG_445D09 line homozygous for the T-DNA insertion was resistant to H. parasitica isolate Waco9, whereas the parental line (Ws-4) was susceptible (FIG. 6A). The At5g24530 transcript could be amplified by RT-PCR using primers in exon 2 and 3 in Ws-4, but not in the homozygous dmr6-2 line (data not shown), indicating that dmr6-2 can be considered a second null-allele.

To corroborate the idea that At5g24530 is required for susceptibility to H. parasitica the dmr6-1 mutant was transformed with the cDNA from At5g24530 cloned under control of the 35S promoter. In five independent dmr6-1 T₂ seedlings the strong overexpression of At5g24530 was confirmed by RT-PCR (data not shown). All T3 lines, homozygous for the transgene, showed restoration of susceptibility to H. parasitica isolate Cala2 (FIG. 6B), confirming that At5g24530 is DMR6. The complementation, together with the identification of two independent dmr6 mutants clearly indicates that a functional DMR6 gene is required for susceptibility to H. parasitica.

DMR6 is transcriptionally activated during H. parasitica infection

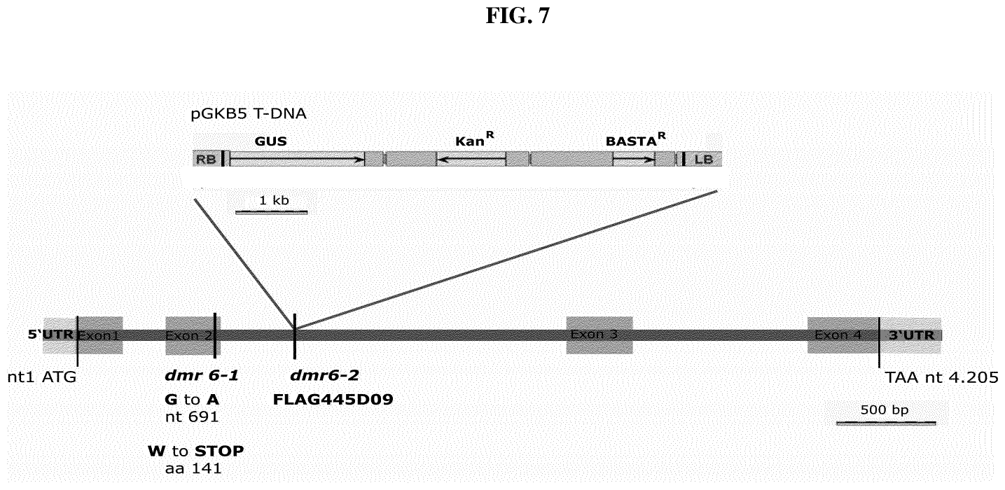

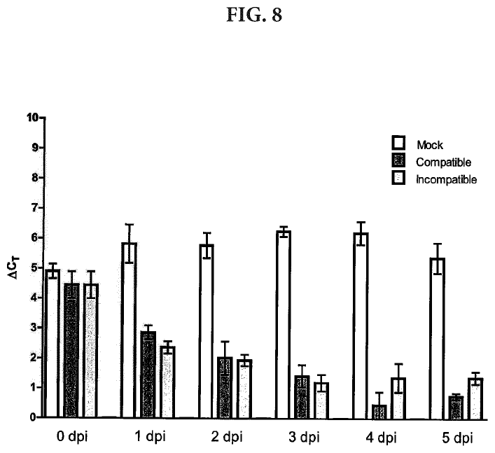

To study the expression of DMR6 during infection with H. parasitica relative transcript levels were measured by quantitative PCR at six different time points from 0 days (2 hours) post inoculation to 5 days post inoculation (dpi) (FIG. 8). RNA was isolated from ten day old Ler seedlings that were spray inoculated with water (mock), compatible, or incompatible H. parasitica isolate. At 2 hours post inoculation (0 dpi) the levels of DMR6 transcripts were equal in the different treatments. Starting from 1 dpi, the level of DMR6 transcript was significantly increased in both the compatible and incompatible interaction compared to mock-treated seedlings. The DMR6 transcript level was slightly but significantly higher at 1 dpi in the incompatible interaction (ACT of 3.5, approximately 11 fold induction) than in the compatible (ΔCT of 3.0, approximately 8 fold induction). The expression level increased further in time to reach a stable high level at 4-5 dpi. At these time points the DMR6 transcript level was higher in the compatible than in the incompatible interaction. The elevated DMR6 transcript levels during compatible and incompatible H. parasitica interactions suggest a role of DMR6 in plant defense. The defense-associated expression of DMR6 could be confirmed in our three enhanced-defense mutants, dmr3, dmr4, and dmr5 (Van den Ackerveken et al., unpublished). Furthermore, in silico analysis of DMR6 levels in the Genevestigator Mutant Surveyor (Zimmermann, P., Hennig, L., and Gruissem,W. (2005). Gene-expression analysis and network discovery using Genevestigator. Trends Plant Sci. 10, 407-409) showed that the gene is strongly induced in the pathogen resistant mutants mpk4 and cpr5. In the cpr5/nprl double mutant the DMR6 transcript level remained high indicating that the induction of DMR6 expression is mostly NPR1 independent. Salicylic acid appears to be an important signal in the induction of DMR6 expression during senescence as nahG transgenic plants (expressing the bacterial salicylate hydroxylase gene) showed only low levels of DMR6 transcript.

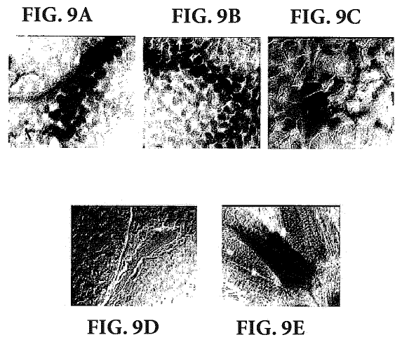

To investigate in more detail how the expression of DMR6 is activated during biotic and abiotic stress, DMR6 reporter lines were generated. The localization of DMR6 expression was studied in transgenic Col-0 and Ler eds1-2 plants containing the DMR6 promoter linked to the uidA ((-glucuronidase, GUS) reporter gene (pDMR6::GUS). To visualize both H. parasitica hyphal growth, by staining with trypan blue, as well as GUS activity, magenta-Xgluc was used as a β-glucuronidase substrate yielding a magenta precipitate. In uninfected plants no GUS expression could be detected in the different plant organelles; roots, meristem, flower, pollen and seed. The expression of DMR6 was induced in the compatible interactions, Ler eds1-2 infected with Cala2 (FIG. 9A), and Col-0 infected with isolate Waco9 (FIG. 9B). GUS expression was also induced in the incompatible interaction Ler eds1-2 inoculated with isolate Emoy2 (FIG. 9C). As shown in FIG. 9A and 9B DMR6 expression was confined to the cells in which H. parasitica had formed haustoria. Plant cells containing the most recently formed haustoria did not show detectable levels of GUS activity (FIG. 9A, indicated by asterisk). During the incompatible interaction (FIG. 9C) activity of the DMR6 promoter could only be detected in the cells that were in contact with the initial invading hyphae. In death cells, resulting from the hypersensitive response (HR, visualized by trypan blue staining indicated in FIG. 9C by asterisk) no GUS activity could be detected, possibly due to protein degradation in these cells. To test if the DMR6 expression in haustoria-containing cells is caused by a wound-like response, seedlings were wound by incision with scissors and stained for GUS activity 3 days later. No detectable promoter DMR6 GUS expression was seen, indicating that the expression of DMR6 is not induced by wounding (FIG. 9D). Furthermore the local induction of DMR6 expression was tested in response to treatment with benzothiadiazole (BTH), a functional analogue of salicylic acid (SA). At 3 days post BTH treatment GUS activity was mainly localized in the newly formed, but not in the mature leaves (FIG. 9E). Analysis of pDMR6::GUS lines confirm the expression data described above and highlights the strictly localized induction of DMR6 in response to H. parasitica infection.

The dmr6-1 mutant constitutively expresses defense associated transcripts

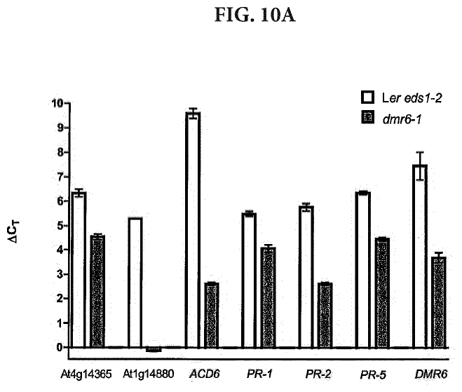

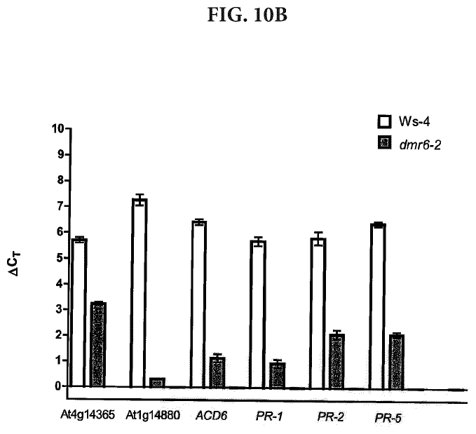

To elucidate how the lack of DMR6 results in H. parasitica resistance, the transcriptome of the dmr6-1 mutant compared to the Ler eds1-2 parental line was analyzed. Probes derived from mRNA of the above-ground parts of 14 day old dmr6-1 and Ler eds1-2 seedlings were hybridized on whole genome CATMA micro arrays. A total of 58 genes were found to be significantly differentially expressed in dmr6-1, of which 51 genes had elevated and 7 genes had reduced transcript levels. A pronounced set of the 51 induced transcripts have been identified as genes associated with activated plant defense responses, e.g., ACD6, PR-5, PR-4/HEL and PAD4. These data indicate that the loss of DMR6 results in the activation of a specific set of defense-associated transcripts. The finding that DMR6 is among the dmr6-1-induced genes corroborates the idea that DMR6 is defense-associated. To test if the induced expression of the defense-associated genes was due to the loss of DMR6 and not due to additional ethane methyl sulfonate (EMS) mutations remaining in the backcrossed dmr6-1 mutant the transcript level of a selection of genes (At4g14365, At1g14880, ACD6, PR-1, PR-2 and PR-5) was verified by quantitative PCR in both the dmr6-1 and dmr6-2 mutant (FIG. 10). We could only test DMR6 transcript levels in the dmr6-1 mutant (FIG. 10A) as the dmr6-2 mutant (FIG. 10B) has a T_DNA insertion disrupting the DMR6 transcript. The induction of DMR6 as observed in the micro array analysis was confirmed by Q-PCR in dmr6-1 compared to Ler eds1-2 (FIG. 10A). FIG. 10A and 10B show that all six selected genes were elevated in both dmr6 mutants compared to the parental lines. The observed elevated expression of the selected defense-associated genes in the dmr6 mutants indicates that lack of DMR6 activates a plant defense response. The activation of this set of defense-associated transcripts could be the primary cause of resistance to H. parasitica in the dmr6 mutants.

### Example 2

### Identification of DMR6 Orthologs in Crops

### 1. Screening of Libraries on the Basis of Sequence Homology

The nucleotide and amino acid sequences of the DMR6 coding sequence and protein of Arabidopsis thaliana are shown in FIG. 2. Public libraries of nucleotide and amino acid sequences were compared with the sequences of FIG. 2. This comparison resulted in identification of the complete DMR6 coding sequences and predicted amino acid sequences in Aquilegia species, Citrus sinensis, Coffea canephora, Cucumis sativus, Gossypium hirsitum, Lactuca sativa, Medicago truncatula, Oryza sativa (3), Populus trichocarpa (2), Solanum lycopersicum (2), Sorghum bicolor, Spinacia oleracea, Vitis vinifera, Zea mays, and Zingiber officinale. The sequence information of the orthologous proteins thus identified is given in Table 1 and visualized in a multiple alignment in FIG. 1. For many other plant species orthologous DNA fragments could be identified by BlastX as reciprocal best hits to the Arabidopsis or other plant DMR6 protein sequences.

2. Identification of Orthologs by means of Heterologous Hybridization

The DMR6 DNA sequence of Arabidopsis thaliana as shown in FIG. 2 is used as a probe to search for homologous sequences by hybridization to DNA of any plant species using standard molecular biological methods. Using this method orthologous genes are detected by southern hybridization on restriction enzyme-digested DNA or by hybridization to genomic or cDNA libraries. These techniques are well known to the person skilled in the art. As an alternative probe the DMR6 DNA sequence of any other more closely related plant species can be used as a probe.

### 3. Identification of Orthologs by Means of PCR

For many crop species, partial DMR6 mRNA or gene sequences are available that are used to design primers to subsequently PCR amplify the complete cDNA or genomic sequence. When 5′ and 3′ sequences are available the missing internal sequence is PCR amplified by a DMR6 specific 5′ forward primer and 3′ reverse primer. In cases where only 5′, internal or 3′ sequences are available, both forward and reverse primers are designed. In combination with available plasmid polylinker primers, inserts are amplified from genomic and cDNA libraries of the plant species of interest. In a similar way, missing 5′ or 3′ sequences are amplified by advanced PCR techniques; 5′RACE, 3′ RACE, TAIL-PCR, RLM-RACE or vectorette PCR.

As an example the sequencing of the Lactuca sativa (lettuce) DMR6 cDNA is provided. From the Genbank EST database at NCBI several Lactuca DMR6 ESTs were identified using the tblastn tool starting with the Arabidopsis DMR6 amino acid sequence. Clustering and alignment of the ESTs resulted in a consensus sequence for a 5′ DMR6 fragment. To obtain the complete lettuce DMR6 cDNA the RLM-RACE kit (Ambion) was used on mRNA from lettuce seedlings. The 3′ mRNA sequence was obtained by using two primers that were designed in the 5′ DMR6 consensus sequence derived from ESTs (Lsat_dmr6_fw_1: CGATCAAGGTCAACACATGG (SEQ ID NO:24), and Lsat_dmr6_fw2: TCAACCATTACCCAGTGTGC) (SEQ ID NO:25) and the 3′RACE primers from the kit. Based on the assembled sequence new primers were designed to amplify the complete DMR6 coding sequence from cDNA to provide the nucleotide sequence and derived protein sequence as presented in FIG. 3.

The complete DMR6 coding sequences from more than 10 different plants species have been identified from genomic and EST databases. From the alignment of the DNA sequences, conserved regions in the coding sequence were selected for the design of degenerate oligonucleotide primers (for the degenerate nucleotides the abbreviations are according to the IUB nucleotide symbols that are standard codes used by all companies synthesizing oligonucleotides; G=Guanine, A=Adenine, T=Thymine, C=Cytosine, R=A or G, Y=C or T, M=A or C, K=G or T, S=C or G, W=A or T, B=C or G or T, D=G or A or T, H=A or C or T, V=A or C or G, N=A or C or G or T).

The procedure for obtaining internal DMR6 cDNA sequences of a given plant species is as follows:
1. mRNA is isolated using standard methods,
2. cDNA is synthesized using an oligo dT primer and standard methods,
3. using degenerate forward and reverse oligonucleotides a PCR reaction is carried out,
4. PCR fragments are separated by standard agarose gel electrophoresis and fragments of the expected size are isolated from the gel,
5. isolated PCR fragments are cloned in a plasmid vector using standard methods,
6. plasmids with correct insert sizes, as determined by PCR, are analyzed by DNA sequencing,
7. Sequence analysis using blastX reveals which fragments contain the correct internal DMR6 sequences,
8. The internal DNA sequence can then be used to design gene- and species-specific primers for 5′ and 3′ RACE to obtain the complete DMR6 coding sequence by RLM-RACE (as described above).

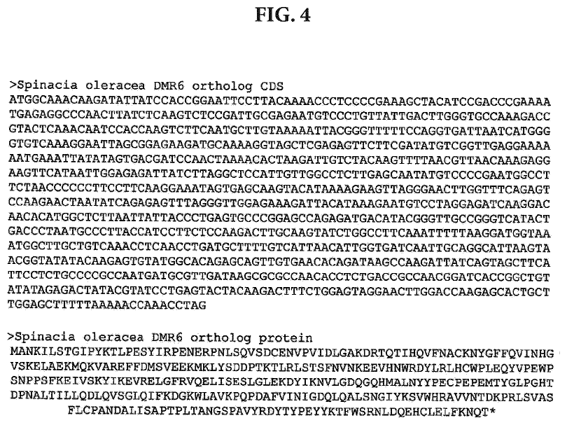

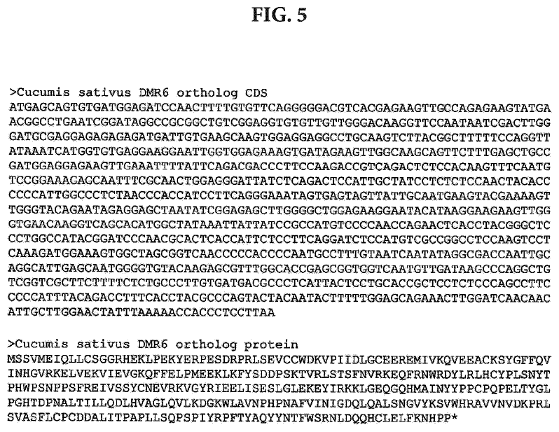

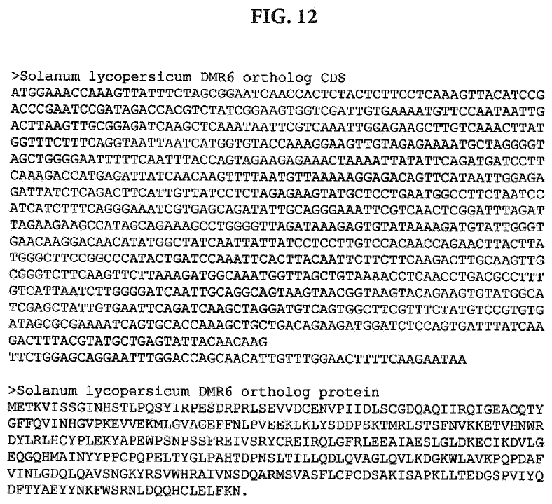

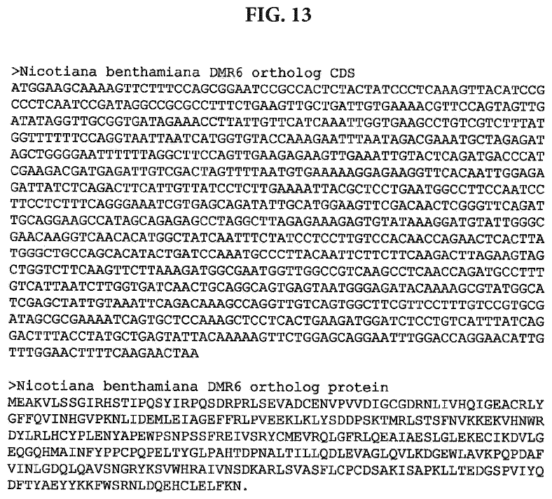

As an example the sequencing of the Cucumis sativus (cucumber) DMR6 cDNA is provided. For cucumber several primer combinations between the following primers were successful in amplifying a stretch of internal coding sequence from cDNA; forward primers dmr6_deg_fw1B (TTCCAGGTDATTAAYCAYGG) (SEQ ID NO:26), dmr6_deg_fw2B CATAAYTGGAGRGAYTAYCT) (SEQ ID NO:27), dmr6_deg_fw3B (GARCAAGGRCARCAYATGGC) (SEQ ID NO:28) and dmr6_deg_fw4 (AATCCTCCTTCHTTCAAGGA) (SEQ ID NO:29) and reverse primers dmr6_deg_rv3B (AGTGCATTKGGGTCHGTRTG) (SEQ ID NO:30), dmr6_deg_rv4 (AATGTTRATGACAAARGCAT) (SEQ ID NO:31) and dmr6_deg_rv5 (GCCATRTGYTGYCCTTGYTC) (SEQ ID NO:32). After cloning and sequencing of the amplified fragments cucumber DMR6-specific primers were designed for 5′ RACE (Cuc_dmr6_rvl: TCCGGACATTGAAACTTGTG (SEQ ID NO:33) and Cuc_dmr6_rv2: TCAAAGAACTGCTTGCCAAC) (SEQ ID NO:34) and 3′ RACE (Cuc_dmr6_fw 1: CGCACTCACCATTCTCCTTC (SEQ ID NO:35) and Cuc_dmr6_fw2: GGCCTCCAAGTCCTCAAAG) (SEQ ID NO:36). Finally the complete cucumber DMR6 cDNA sequence was amplified and sequenced (FIG. 5). A similar approach was a used for spinach, Spinacia oleracea (FIG. 4), Solanum lycopersicum (FIG. 12) and Nicotiana benthamiana (FIG. 13).

Orthologs identified as described in this example can be modified using well-known techniques to induce mutations that reduce the DMR6 expression or activity, to obtain non-genetically modified plants resistant to Fungi or Oomycota. Alternatively, the genetic information of the orthologs can be used to design vehicles for gene silencing, and to transform the corresponding crop plants to obtain plants that are resistant to Oomycota.

### Example 3

### Mutation of Seeds

Seeds of the plant species of interest are treated with a mutagen in order to introduce random point mutations in the genome. Mutated plants are grown to produce seeds and the next generation is screened for the absence of reduction of DMR6 transcript levels or activity. This is achieved by monitoring the level of DMR6 gene expression, or by searching for nucleotide changes (mutations) by the TILLING method, by DNA sequencing, or by any other method to identify nucleotide changes. The selected plants are homozygous or are made homozygous by selfing or inter-crossing. The selected homozygous plants with absent or reduced DMR6 transcript activity are tested for increased resistance to the pathogen of interest to confirm the increased disease resistance.

### Example 4

### Transfer of a Mutated Allele into the Background of a Desired Crop

Introgression of the desired mutant allele into a crop is achieved by crossing and genotypic screening of the mutant allele. This is a standard procedure in current-day marker assistant breeding of crops.

### Example 5

### Use of the DMR6 Promotor for Pathogen-Induced Gene Expression and the Generation of Disease Resistant Plants

Precise control of transgene expression is pivotal to the engineering of plants with increased disease resistance. In the past, constitutive overexpression of transgenes frequently has resulted in poor quality plants. It has therefor been suggested to use pathogen-inducible promotors, by which the transgenes are expressed only when and where they are needed—at infection sites.

Local and inducible expression of engineered genes, e.g., master switch genes, elicitor or Avr genes, anti-microbial genes, or toxic genes, results in the activation of defense or cell death that will lead to pathogen resistance, such as described by Gurr and Rushton (Trends in Biotechnology 23: 275-282, 2005). A good example is provided by De with (Annu. Rev. Phytopathol. 30: 391-418, 1992) who proposes the use of the Avr9-Cf9 combination to achieve induced cell death leading to disease resistance. The tissue-specificity and inducibility of expression is of prime importance for such approaches, as described by Gurr and Rushton (Trends in Biotechnology 23: 283-290, 2005).

According to the present disclosure, the DMR6 promoter has been demonstrated to show a strong, inducible, localized expression based on promoter-GUS analysis. Thus, the DMR6 promotor is very suitable for engineering disease resistance in transgenic plants. The DMR6 promoter consists of a region of 2.5 kb that is upstream of the Arabidopsis DMR6 coding sequence

(ATG start codon) and includes the 5′UTR (as depicted in FIG. 11). This pathogen-inducible promotor is then used to engineer suitable transgene constructs, using standard techniques known the person skilled in the art.

Using orthologous DNA sequences from a given plant species primers are designed for PCR. These are then used to screen genomic libraries of the plant species of interest to identify the genomic clones that contain the DMR6 ortholog with its promoter and regulatory sequences. Alternatively, the genomic clones are isolated by screening a library with a labelled PCR fragment corresponding to the DMR6 orthologous gene. Sequencing reveals the nucleotide sequence of the promoter. The region of 2-5 kb upstream the DMR6 orthologous coding sequence (ATG start codon), so including the 5′UTR, is then amplified by PCR to engineer transgene constructs for plant transformation.

### Example 6

This example demonstrates the complementation of mutant dmr6-1 in Arabidopsis thaliana by DMR6 orthologs from 4 different crop species. For this, DMR6 orthologs of Cucumis sativa (Cs), Spinacia oleracea (So), Lactuca sativa (Ls) and Solanum lycopersicum (Sl) were cloned into a plant expression vector under the control of the 35S promoter and, subsequently, this vector was transformed into a Arabidopsis thaliana mutant dmr6-1.

Briefly, mRNA was isolated using standard methods and cDNA was synthesized using an oligo dT primer and standard methods. Subsequently, PCR fragments were generated using primer pairs for each crop as depicted in table 3 below. The generated PCR products were cloned into a pENTR/D-TOPO vector using the pENTR/D-TOPO cloning kit from Invitrogen and resulting plasmids with correct insert sizes, as determined by PCR, were analyzed by DNA sequencing. Recombination to the pB7WG2,0 vector was done using LR clonase II from Invitrogen and the resulting plasmids were analyzed by PCR and digestion with restriction enzymes. Suitable plasmids were transformed into Agrobacterium tumefaciens C58C1 PGV2260 and plasmids from Agrobacterium were analyzed by PCR and digestion with restriction enzymes.

Arabidopsis thaliana dmr6-1 plants were transformed with the above constructs by dipping into Agrobacterium solution and overexpression of crops DMR6 in Arabidopsis T1 plants is verified by RT-PCR using the crops DMR6 cloning primers (Table 3). Finally, Arabidopsis T2 and T3 plants were infected with Hyaloperonospora parasitica Cala2 to confirm complementation.

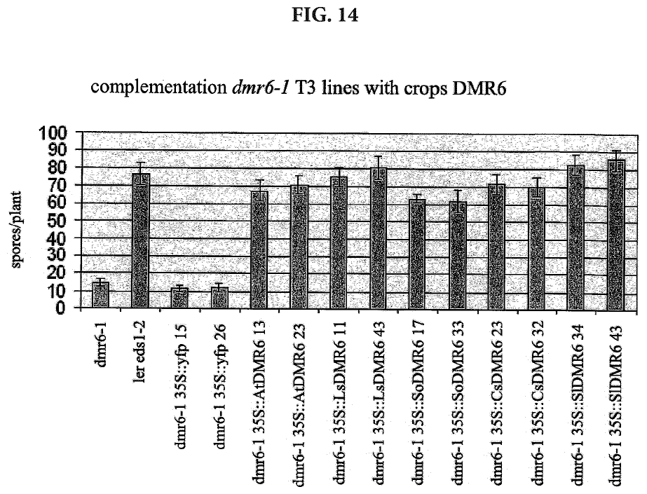

The results are shown in FIG. 14.

As shown in FIG. 14, all DMR6 orthologs tested were capable of complementing Arabidopsis thaliana mutant dmr6-1 indicating that the DMR6 orthologs identified encode DMR6 proteins with a similar functionality as Arabidopsis thaliana DMR6.

### Example 7

Soybean plants are transformed with constructs, either for providing over expression of each of the genes encoding DMR6 proteins SEQ ID NO:115 and SEQ ID NO:116, or for providing silencing each of the genes encoding DMR6 proteins SEQ ID NO:115 and SEQ ID NO:116.

Soybean silencing constructs are generated using Gateway cloning of a 300 bp fragment identical to the middle part of the CDS of each of the genes.

SoybeanDMR6 Sequences:

SEQ ID NO: 115
MDIKVLSSGVQYSNLPESYIRPESERPRLSEVSECEDVPI
IDLGSQNRAQIVHQIGEACRNYGFFQVINHGVALEAAKEM
EEVAHGFFKLPVEEKLKLYSEDTSKTMRLSTSFNVKKETVRNVVRDYLRLHCYPLEKYAPEWPSNPPSFKETVTEYCTII
RELGLRIQEYISESLGLEKDYIKNVLGEQGQHMAVNYYPP
CPEPELTYGLPGHTDPNALTILLQDLQVAGLQVLKDGKWL
AVSPQPNAFVINIGDQLQALSNGLYKSVWHRAVVNVEKPR
LSVASFLCPNDEALISPAKPLTEHGSEAVYRGFTYAEYYK
KFWSRNLDQEHCLELFKNK
SEQ ID NO: 116
MDTKVLSSGVQYSNLPESYIRPESERPRLSEVSECEDVPI
IDLGCQNRAQIVHQIGEACRNYGFFQVINHGVALEAAKEM
AEVAHGFFKLPVEEKLKLYSEDPSKTMRLSTSFNVKKETV
HNVVRDYLRLHCYPLDKYAPEWPSNPPSFKETVTEYCTLV
RELGLRIQEYISESLGLEKDYIKNVLGEQGQHMAVNYYPP
CPEPELTYGLPGHTDPNALTILLQDLQVCGLQVLKNGKWL
AVNPQPNAFVINIGDQLQALSNGLYKSVWHRAVVNVEKPR
LSVASFLCPNDEALISPAKPLTEGGSEAIYRGFTYAEYYK
KFWSRNLDQEHCLEFFKNK

The generated ENTRY vectors are Gateway cloned into the pHellsgatel2 binary vector. Following Agrobacterium transformation according standard procedure for soybean. The silencing constructs are able to silence each of the genes encoding SEQ ID Nos: 115 and 116. At least six independent T1 transformants are maintained for each construct.

Offspring from transformed soybean plants are subjected to a disease test by inoculation of Phytophthora sojae into the soil as described by Jiang (Phytopathology 107: 216-223, 2017). In short, the procedure includes mixing the P. sojae inoculum into the soil and evaluating the survival rate of the soybean seedlings 12 to 16 dpi. The plants are visually analyzed by scoring root rot severity on a visual scale from 1 to 9, wherein 1 means resistance and 9 means susceptible. Approximately 10-15 seedlings are used per inoculation.

Phytophthora sojae Assay details

A standard wounded hypocotyl method is used to evaluate disease from T1 (first generation transgenic) plants. In short, an incision in the hypocotyl below the node is made and the wound is covered with inoculum. The plants are placed in a tray with 100% RH at 25° C. for 48 hours following transfer to a growth chamber. Six days post inoculation, plants are visually scored for disease symptoms as percentage of surviving seedlings (minimal 20 seedlings per assay) according Sugano (Plant Pathology 62: 1048-1056, 2013).

### TABLES

Table 1 lists the GI numbers (GenInfo identifier) and Genbank accession number for Expressed Sequence Tags (ESTs) and mRNA or protein sequences of the Arabidopsis DMR6 mRNA and orthologous sequences from other plant species. A GI number (genlnfo identifier, sometimes written in lower case, “gi”) is a unique integer which identifies a particular sequence.

The GI number is a series of digits that are assigned consecutively to each sequence record processed by NCBI. The GI number will thus change every time the sequence changes. The NCBI assigns GI numbers to all sequences processed into Entrez, including nucleotide sequences from DDBJ/EMBL/GenBank, protein sequences from SWISS-PROT, PIR and many others. The GI number thus provides a unique sequence identifier which is independent of the database source that specifies an exact sequence. If a sequence in GenBank is modified, even by a single base pair, a new GI number is assigned to the updated sequence. The accession number stays the same. The GI number is always stable and retrievable. Thus, the reference to GI numbers in the table provides a clear and unambiguous identification of the corresponding sequence.)

TABLE 1

| Species | Common name | Detail | GI number | Genbank |
|---|---|---|---|---|
| Arabidopsis thaliana | Thale cress | mRNA | 42568064 | NM_122361 |
| Aquilegia_sp | Aquilegia | ESTs | 75461114 | DT768847.1 |
| | | | 74538666 | DT745001.1 |
| | | | 74562677 | DT760187.1 |
| | | | 75461112 | DT768846.1 |
| | | | 74562675 | DT760186.1 |
| Citrus sinensis | Sweet Orange | ESTs | 5793134 | CX672037.1 |
| | | | 57933368 | CX673829.1 |
| | | | 63078039 | CX309185.1 |
| Coffea canephora | Coffea | ESTs | 82485203 | DV705375.1 |
| | | | 82458236 | DV684837.1 |
| | | | 82461999 | DV688600.1 |
| | | | 82487627 | DV707799.1 |
| Gossypium hirsutum | Cotton | ESTs | 109842586 | DW241146.1 |
| | | | 48751103 | CO081622.1 |
| Sorghum bicolor | Sorghum | ESTs | 45992638 | CN150358.1 |
| | | | 57813436 | CX614669.1 |
| | | | 45985339 | CN145819.1 |
| | | | 57821006 | CX622219.1 |
| | | | 45989371 | CN148311.1 |
| | | | 57821495 | CX622708.1 |
| | | | 45959033 | CN130459.1 |
| | | | 45985193 | CN145752.1 |
| | | | 18058986 | BM322209.1 |
| | | | 45958822 | CN130381.1 |
| | | | 30164583 | CB928312.1 |
| Medicago truncatula | Barrel medic | Genome | | MtrDRAFT_AC119415g1v1 |
| | | draft | | |
| | | protein | 92878635 | ABE85154 |
| Oryza sativa 1 | Rice | Genome | | OSJNBb0060I05.4 |
| | | protein | 18057095 | AAL58118.1 |
| Oryza sativa 2 | | mRNA | 115450396 | NM_001055334 |
| | | protein | 115450397 | NP_001048799 |
| Oryza sativa 3 | | mRNA | 115460101 | NM_001060186 |
| | | protein | 115460102 | NP_001053651 |
| Populus trichocarpa 1 | Poplar | Genome: LG_XII: 3095392-3103694 | | |
| | | protein: Poptr1_1: 569679, | | |
| | | eugene3.00120332 | | |
| Populus trichocarpa 2 | Poplar | Genome: LG_XV: 201426-209590 | | |
| | | protein: Poptr1_1: 732726, | | |
| | | estExt_Genewise1_v1.C_LG_XV0083 | | |
| Solarium lycopersicum 1 | Tomato | ESTs | 62932307 | BW689896.1 |
| | | | 58229384 | BP885913.1 |
| | | | 117682646 | DB678879.1 |
| | | | 5894550 | AW035794.1 |
| | | | 117708809 | DB703617.1 |
| | | | 62934028 | BW691617.1 |
| | | | 15197716 | BI422913.1 |
| | | | 4381742 | AI486371.1 |
| | | | 5601946 | AI896044.1 |
| | | | 4387964 | AI484040.1 |
| | | | 4383017 | AI487646. |
| | | | 5278230 | AI780189.1 |
| | | | 12633558 | BG133370.1 |
| | | | 76572794 | DV105461.1 |
| | | | 117692514 | DB718569.1 |
| | | | 4385331 | AI489960.1 |
| | | | 4383253 | AI487882.1 |
| | | | 4384827 | AI489456.1 |
| Solarium lycopersicum 2 | Tomato | ESTs | 47104686 | BT013271.1 |
| | | | 14685038 | BI207314.1 |
| | | | 14684816 | BI207092.1 |
| Zea mays | Maize | ESTs | 110215403 | EC897301.1 |
| | | | 76291496 | DV031064.1 |
| | | | 91050479 | EB160897.1 |
| | | | 91874282 | EB404239.1 |
| | | | 110540753 | EE044673.1 |
| | | | 78111856 | DV530253.1 |
| | | | 94477588 | EB706546.1 |
| | | | 71441483 | DR822533.1 |
| | | | 78111699 | DV530096.1 |
| | | | 78107139 | DV525557.1 |
| | | | 76017449 | DT944619.1 |
| | | | 91048249 | EB158667.1 |
| | | | 78104908 | DV523326.1 |
| | | | 78088214 | DV516607.1 |
| | | | 76291495 | DV031063.1 |
| | | | 71441482 | DR822532.1 |
| | | | 78088213 | DV516606.1 |
| Vitis vinifera | Grape | ESTs | 33396402 | CF202029.1 |
| | | | 33399765 | CF205392.1 |
| | | | 45770972 | CN006824.1 |
| | | | 45770784 | CN006636.1 |
| | | | 45770528 | CN006380.1 |
| | | | 45770631 | CN006483.1 |
| | | | 33400623 | CF206250.1 |
| | | | 33396335 | CF201962.1 |
| | | | 30134763 | CB920101.1 |
| | | | 30305300 | CB982094.1 |
| | | | 71857419 | DT006474.1 |
| | | | 30305235 | CB982029.1 |
| Zingiber officinale | Ginger | ESTs | 87108948 | DY375732.1 |
| | | | 87095447 | DY362231.1 |
| | | | 87095448 | DY362232.1 |
| | | | 87094804 | DY361588.1 |
| | | | 87095449 | DY362233.1 |
| | | | 87094803 | DY361587.1 |

| | | |
|---|---|---|
| Lactuca sativa | Lettuce | Sequence described in this patent |
| | | application |
| Spinacia oleracea | Spinach | Sequence described in this patent |
| | | application |
| Cucumis sativus | Cucumber | Sequence described in this patent |
| | | application |
| Nicotiana benthamiana | Tabac | Sequence described in this patent |
| | | application |

Table 2 lists primer sequences of insertion/deletion markers (size difference in brackets) used in the mapping and cloning of the DMR6 gene.

TABLE 2

| Name | | INDEL/ | Forward | Reverse |
|---|---|---|---|---|
| primer | Gene | enzyme | primer | primer |
| IND_MOP9 | At5G24210 | | tttggga | catattc |
| | | | acagaaa | aaaaggga |
| | | | aagttgg | aaatccca |
| | | | aggt | ga |
| | | | (SEQ ID | (SEQ ID |
| | | | NO: 37) | NO: 38) |
| IND_Kl6H17 | At5g24420 | | tggggtt | tggccaat |
| | | | gtggttt | agtagttg |
| | | | attctgt | atacgcaa |
| | | | tgac | ga |
| | | | (SEQ ID | (SEQ ID |
| | | | NO: 39) | NO: 40) |
| IND_T4C12 | At5g24820 | | tctcgg | tattcca |
| | | | gtaaga | acttgcg |
| | | | cacaag | acgtaga |
| | | | tcgagat | gcat |
| | | | (SEQ ID | (SEQ ID |
| | | | NO: 41) | NO: 42) |
| IND_T11H3 | At5g24950-60 | | ccaattg | cggcttt |
| | | | ggttatt | taacaac |
| | | | tacttcg | atatttt |
| | | | att | cca |
| | | | (SEQ ID | (SEQ ID |
| | | | NO: 43) | NO: 44) |
| IND_F21J6 | At5g25270 | | aacacat | cctctgcc |
| | | | caccaag | ccaagaaa |
| | | | atgaatc | tattgaga |
| | | | caga | t |
| | | | (SEQ ID | (SEQ ID |
| | | | NO: 45) | NO: 46) |
| M450 | At5G24450 | 18 | agctttg | gcggtat |
| | | | tatggta | acggggg |
| | | | gtgccaa | ttaaaat |
| | | | tga | cta |
| | | | (SEQ ID | (SEQ ID |
| | | | NO: 47) | NO: 48) |
| M490 | At5g24490 | TaqI | atggcca | acaagcaa |
| | | | accactc | gaagaaca |
| | | | tttgtta | gcgaag |
| | | | c | (SEQ ID |
| | | | (SEQ ID | NO: 50) |
| | | | NO: 49) | |
| M525 | At5g24520-30 | TaqI | gaaattt | tcaagatc |
| | | | ggttgtt | ttcatatt |
| | | | ggcattt | ctcattcc |
| | | | atc | a |
| | | | (SEQ ID | (SEQ ID |
| | | | NO: 51) | NO: 52) |
| M545 | At5G24540/50 | 41 | cagctga | cttgcaat |
| | | | agtatgt | tgttggga |
| | | | ttcatcc | ctaggta |
| | | | cttt | a |
| | | | (SEQ ID | (SEQ ID |
| | | | NO: 53) | NO: 54) |
| M555 | At5G24550/60 | 14 | tcactaa | tatacagc |
| | | | ccagtga | gaatagca |
| | | | aaaaggt | aagccaag |
| | | | tgc | (SEQ ID |
| | | | (SEQ ID | NO: 56) |
| | | | NO: 55) | |
| M470 | At5g24470 | HphI | ccgcgag | cagtttaa |
| | | | tgtaata | cgcatgaa |
| | | | tatctct | gtgctagt |
| | | | cct | (SEQ ID |
| | | | (SEQ ID | NO: 58) |
| | | | NO: 57) | |
| M590 | At5g24590 | PdmI | gcatcat | tagtggat |
| | | | ttgtacc | actctgtc |
| | | | gtactga | cctgaggt |
| | | | gtc | (SEQ ID |
| | | | (SEQ ID | NO: 60) |
| | | | NO: 59 | |

Table 3 lists primer pairs for cloning dmr6 orthologs in a suitable plant expression vector

TABLE 3

| | | |
|---|---|---|
| Arabidopsis | AtDMR6_fw | CACCATGGCGGCAAA |
| thaliana | | GCTGATA |
| | | (SEQ ID NO: 85) |
| | AtDMR6UTR_rv | GACAAACACAAAGGC |
| | | CAAAGA |
| | | (SEQ ID NO: 86) |
| Cucumis | cuc_fw | CACCATGAGCAGTGT |
| sativa | | GATGGAGAT |
| | | (SEQ ID NO: 87) |
| | cucUTR_ry | TGGGCCAAAAAGTT |
| | | TATCCA |
| | | (SEQ ID NO: 88) |
| Spinacia | spi_fw | CACCATGGCAAACAA |
| oleracea | | GATATTATCCAC |
| | | (SEQ ID NO: 89) |
| | spiUTR_ry | TTGCTGCCTACAAAA |
| | | GTACAAA |
| | | (SEQ ID NO: 90) |
| Lactuca | Lsat_fw | CACCATGGCCGCAAA |
| sativa | | AGTCATCTC |
| | | (SEQ ID NO: 91) |
| | LsatUTR_ry | CATGGAAACACATAT |
| | | TCCTTCA |
| | | (SEQ ID NO: 92) |
| Solanum | Slyc1dmr6_fw | CACCATGGAAACCAA |
| lycopersicum | | AGTTATTTCTAGC |
| | | (SEQ ID NO: 93) |
| | Slyc1dmr6UTR_ | GGGACATCCCTATGA |
| | rv | ACCAA |
| | | (SEQ ID NO: 94) |

1-9. (canceled)

10. A Phytophthora sojae resistant soybean plant, wherein the soybean plant has reduced activity of a first DMR6 protein comprising amino acid sequence SEQ ID NO:116 as compared to the activity of the first DMR6 protein in a corresponding soybean plant that is not resistant to Phytophthora sojae.

11. The soybean plant of claim 10, wherein the reduced activity of the first DMR6 protein is the result of a non-natural mutation in a first gene encoding the first DMR6 protein, and wherein the non-natural mutation results in reduced expression or reduced transcription of the first gene.

12. A seed, tissue, or plant part of the soybean of claim 11, wherein the seed, tissue, or plant part comprises the non-natural mutation in the first gene and has a reduced activity of the first protein.

13. The soybean plant of claim 10, wherein the soybean plant further has a reduced activity of a second DMR6 protein comprising amino acid sequence SEQ ID NO:115 as compared to the activity of the second DMR6 protein in a corresponding soybean plant that is not resistant to Phytophthora sojae.

14. The soybean plant of claim 13, wherein the reduced activity of the first DMR6 protein is the result of a non-natural mutation in a first gene encoding the first DMR6 protein, wherein the non-natural mutation results in reduced expression or reduced transcription of the first gene; and wherein the reduced activity of the second DMR6 protein is the result of a non-natural mutation in a second gene encoding the second DMR6 protein, wherein the non-natural mutation results in reduced expression or reduced transcription of the second gene.

15. A seed, tissue, or plant part of the soybean plant of of claim 13, wherein the seed, tissue, or plant part comprises the non-natural mutation in the first gene and the non-natural mutation in the second gene, and wherein the seed, tissue, or plant part comprises the reduced activity of the first DMR6 protein and the reduced activity of the second DMR6 protein.

16. A method for obtaining a Phytophthora sojae resistant soybean plant comprising reducing an endogenous activity of a first DMR6 protein comprising amino acid sequence SEQ ID NO:116 in a soybean plant as compared to the activity of the first DMR6 protein in a corresponding soybean plant that is not resistant to Phytophthora sojae.

17. The method of claim 16, wherein the endogenous activity of the first DMR6 protein is reduced by introducing a non-natural mutation in a first gene encoding the first DMR6 protein, wherein the introduction of the non-natural mutation results in reduced expression or reduced transcription of the first gene.

18. A Phytophthora sojae resistant soybean plant produced by the method of claim 16, wherein the soybean plant comprises the non-natural mutation in the first gene and has reduced activity of the first DMR6 protein.

19. A seed, tissue, or plant part of the soybean plant of of claim 18, wherein the seed, tissue, or plant part comprises the non-natural mutation in the first gene and has reduced activity of the first DMR6 protein.

20. The method of claim 16, further comprising reducing an endogenous activity of a second DMR6 protein comprising amino acid sequence SEQ ID NO:115 in a soybean plant as compared to the activity of the second DMR6 protein in a corresponding soybean plant that is not resistant to Phytophthora sojae.

21. The method of claim 20, wherein the endogenous activity of the first DMR6 protein is reduced by introducing a non-natural mutation in a first gene encoding the first DMR6 protein of SEQ ID NO:116, wherein the introduction of the non-natural mutation results in reduced expression or reduced transcription of the first gene; and wherein the endogenous activity of the second DMR6 protein is reduced by introducing a non-natural mutation in a second gene encoding the second DMR6 protein of SEQ ID NO:115, wherein the introduction of the non-natural mutation results in reduced expression or reduced transcription of the second gene.

22. A Phytophthora sojae resistant soybean plant produced by the method of claim 21, wherein the soybean plant comprises the non-natural mutation in the first gene and the non-natural mutation in the second gene and has reduced activity of the first DMR6 protein and reduced activity of the second DMR6 protein.

23. A seed, tissue, or plant part of the soybean plant of of claim 22, wherein the seed, tissue, or plant part comprises the non-natural mutation in the first gene and the non-natural mutation in the second gene and has reduced activity of the first DMR6 protein and reduced activity of the second DMR6 protein.
